(19) United States
(12) Patent Application Publication
(10) Pub. No.: US 2012/0045526 A1
(43) Pub. Date: Feb. 23, 2012

(54) PHARMACEUTICAL COMPOUND WHICH INCLUDES CLINOPTILOLITE

(76) Inventor: Kevin Gast, Centurion (ZA)

(21) Appl. No.: 13/117,298

(22) Filed: May 27, 2011

Related U.S. Application Data
(63) Continuation of application No. PCT/IB2009/055382, filed on Nov. 27, 2009.

(30) Foreign Application Priority Data
Nov. 28, 2008 (ZA) 2008/10136

(51) Int. Cl. A61K 33/06; A61P 1/00; A61P 25/32; A61P 1/04

(52) U.S. Cl. 424/684

(57) ABSTRACT

This invention is for a compound for treating a human or animal body to relieve the symptoms of any one of chemical-, substance-, and medicine induced gastrointestinal tract irritation, the compound including clinoptilolite. The invention is also for a compound for treating a human or animal body to lower the incidences of gastic events in persons using non-steroidal, anti-inflammatory medications, the compound including clinoptilolite.

### CROSS REFERENCE TO RELATED APPLICATIONS

THE present application is a continuation of International Application No. PCT/1B2009/055382, filed on Nov. 27, 2009, which claims priority to South African Application No. 2008/10136, filed on Nov. 28, 2008, the contents of each of which is incorporated herein by reference.

### FIELD OF THE INVENTION

THIS INVENTION relates to a pharmaceutical compound which includes clinoptilolite, as well as to a method of enhancing the efficacy of clinoptilolite in sequestering harmful compounds. More specifically, the invention relates to the use of potentiated clinoptilolite in treating various medical conditions in humans.

### BACKGROUND TO THE INVENTION

Zeolites are a group of naturally occurring and synthetic microporous, crystalline, hydrated aluminosilicates. Zeolites have well defined chemical structures containing AlO₄ and SiO₄ moieties linked through a common oxygen atom. The applicant is aware of many uses of zeolites in both industry and agriculture which is due to their three-dimensional structure, which endows them with specific physicochemical properties including: ion exchange capacity, adsorbent nature, size exclusion framework as well as catalytic properties.

Clinoptilolite, colloquially known as clinoptolite, is a geological term used to describe one of the naturally occurring, high silica content zeolites. The functional physicochemical attributes of clinoptilolite in its natural state allow for its usage in many applications such as in chemical sieve applications, gas adsorption applications, as feed additives, as food additives, odour control agents, and as a water filter for municipal and residential drinking water.

Additionally, clinoptilolite in its natural state is known to exhibit diverse biological activities and may be of clinical use as an adjuvant to anticancer therapy, as a vaccine adjuvant, as a glucose adsorbent for the treatment of diabetes, as an antioxidant, and as a regulator of the immune system.

Clinoptilolite in its naturally occurring state is not always ideal for use as an adsorption-, sieving-, or sequestering agent, as it frequently has varying amounts of contaminants associated therewith which tend to impede the adsorptive-, sieving-, and/or sequestering capabilities thereof. Such contaminants also tend to impede the cation exchange capabilities of naturally occurring clinoptilolite.

The applicant has invented a method to potentiate clinoptilolite in order to increase the adsorptive-, sieving-, sequestering-, and/or cation exchange capabilities of naturally occurring clinoptilolite. Further research has determined that by manipulating the particle sizes of the potentiated clinoptilolite further ailments and medical conditions can be treated. It is the object of this invention to potentiate clinoptilolite using different particle sizes in order to treat a wide spectrum of medical conditions.

### SUMMARY OF THE INVENTION

According to one aspect of the invention, there is provided a method of potentiating clinoptilolite, the method including the steps of:

providing any one of fresh and spent clinoptilolite;

exposing the clinoptilolite to a saline solution having a sodium ion content of between 0.1% and 60% to liberate impurities from the clinoptilolite; and

drying the washed clinoptilolite fraction to render a potentiated clinoptilolite fraction.

The clinoptilolite may be exposed to the saline solution for a period of between 0.1 hours and 100 days. More specifically, the clinoptilolite may be exposed to the saline solution for a period of between 12 hours and 72 hours. In particular, the clinoptilolite may be exposed to the saline solution for a period of about 48 hours. This allows an optimal cation exchange capacity of the clinoptilolite to be reached.

The saline solution may have a sodium ion content of between 5% and 20%. More specifically, the saline solution may have a sodium ion content of about 10%.

The clinoptilolite may be exposed to the saline solution in a ratio of between 1:1 and 1:100 clinoptilolite:saline solution. More specifically, the clinoptilolite may be exposed to the saline solution in a ratio of 1:1 and 1:10.

The washed clinoptilolite may be dried by heated air. Preferably, the heated air may be filtered. The air may be heated to a temperature of between 40° C. and 450° C. More specifically, the air may be heated to a temperature of 80° C. and 120° C. Preferably, the air may be heated to a temperature of about 100° C. Typically, drying may occur in an encapsulated or sealed liquidized bed reactor. Alternatively, drying may also be accomplished under a vacuum, which the applicant has found increases the cationic exchange capacity of the clinoptolite.

The method may include the prior step of grinding the clinoptilolite to an average particle size of between 0.1 μm and 3 mm, depending on the intended use of the clinoptilolite. More particularly, the potentiated clinoptilolite may comprise particles with an average particle size of less than 2 mm, preferably less than 1.8 mm, more preferably less than 1 mm. The method may therefore include grinding the clinoptilolite to a size of between 0.1 mm and 1 mm. More specifically, the clinoptilolite may be ground to a size of about 0.5 mm. It is to be understood that the smaller the average size of the clinoptilolite particles, the larger the available reaction surface is per particle for reacting with compounds which are to be adsorbed or sequestered by the clinoptilolite.

The clinoptilolite may be bulked in bags prior to washing. Fortuitously, the method of the invention allows clinoptilolite to be processed in bulk, by allowing bags to be processed in large holding baths, typically 20 ton holding baths.

The method may include the further step of rinsing the clinoptilolite after the clinoptilolite has been exposed to the saline solution. The rinsing is for removing excess sodium, prior to drying thereof.

According to another aspect of the invention, there is provided a pharmaceutical compound, which includes potentiated clinoptilolite produced according to a method as described.

According to another aspect of the invention, there is provided a medicament, which includes potentiated clinoptilolite produced according to a method as described.

The medicament may include potentiated clinoptilolite in particulate form, although it may also be in the form of a tablet or capsule.

According to another aspect of the invention, there is provided a use of potentiated clinoptilolite produced according to a method as described, in the manufacture of a pharmaceutical compound or for preparing a medicament.

According to another aspect of the invention there is provided a use of a pharmaceutical compound or a medicament, as described, for treating a condition selected from the group consisting of medication overdose, heavy metals poisoning, nitrates poisoning, alcohol poisoning, alcoholic keto-acidosis, gout, said pharmaceutical compound or medicament including clinoptilolite potentiated using the method, as described, to improve or change or enhance the cation exchange capacity of the clinoptilolite and said use including administering an effective dose of the pharmaceutical compound or medicament to a patient in need thereof.

According to another aspect of the invention, there is provided a use of a pharmaceutical compound or a medicament, as described, to absorb any one of metals and nitrates from linings of the alimentary canal or digestive tract.

According to another aspect of the invention, there is provided a use of a pharmaceutical compound or a medicament, as described, to absorb any one of metals and nitrates from linings of the mouth, pharynx, oesophagus, stomach and intestine.

According to another aspect of the invention, there is provided a use of a pharmaceutical compound or medicament, as described, to absorb metals selected from the group: Pb, Cs, Rb, K, Ba, Sr, Zn, Cu, Co, Ni, Hg, Fe, Al, Li, and nitrates, while simultaneously releasing calcium and magnesium, which can be beneficial to the human or animal body.

While many substances, such as metals and nitrates, can be absorbed from any part of the alimentary canal, it is believed that excess amounts of these substances, which can function as impurities or toxins to the body when present in excess amounts, will in particular be absorbed from the linings of the stomach and intestine.

The substances, such as metals and/or nitrates, which are adsorbed to or sequestered by the potentiated clinoptilolite, are removed from the body during faecal passage.

According to another aspect of the invention, there is provided a method of treating a human or animal body, which includes

orally administering to a human or animal an effective quantity of potentiated clinoptilolite produced according to the method as described; and

permitting the potentiated clinoptilolite to absorb any one of metals and nitrates from linings of the alimentary canal or digestive tract while in contact with these linings.

The clinoptilolite may be administered at a dosage rate of between 100 mg and 6 g per dose. The clinoptilolite may be administered between once to three times daily. The clinoptilolite may be administered at a dosage rate of about 1.5 g, twice daily. The potentiated clinoptilolite may be administered in the form of a suspension in a liquid, such as water.

The potentiated clinoptilolite may be in particulate form, although it may also be in the form of a tablet or capsule.

According to another aspect of the invention, there si provided a method of treating a human or animal body, which includes using clenoptilolite to relieve the symptoms of any one of chemical-, substance-, and medicine induced gastrointestinal tract irritation including but not limited to diarrhea, heartburn, erosions, vomiting, nausea, discomfort and pain.

According to another aspect of the invention, there is provided a use of a medicament for treating a human or animal body to relieve the symptoms of any one of chemical-, substance-, and medicine induced gastrointestinal tract irritation, the medicament including clinoptilolite.

According to another aspect of the invention, there is provided a compound for treating a human or animal body to relieve the symptoms of any one of chemical-, substance-, and medicine induced gastrointestinal tract irritation, the compound including clinoptilolite.

According to another aspect of the invention, there is provided a method of treating a human or animal body, which includes using clenoptilolite to reduce ancillary symptoms such as headache, photophobia, nausea and other symptoms associated with intoxications such as alcohol intoxication.

According to another aspect of the invention, there is provided a use of a medicament for treating a human or animal body to reduce ancillary symptoms associated with intoxications, the medicament including clinoptilolite.

According to another aspect of the invention, there is provided a compound for treating a human or animal body to reduce ancillary symptoms associated with intoxications, the compound including clinoptilolite.

According to another aspect of the invention, there is provided a method of treating a human or animal body, which includes using clenoptilolite to lower the incidences of gastric events in persons using non-steroidal, anti-inflammatory medications.

According to another aspect of the invention, there is provided a use of a medicament for treating a human or animal body to lower the incidences of gastric events in persons using non-steroidal, anti-inflammatory medications, the medicament including clinoptilolite.

According to another aspect of the invention, there is provided a compound for treating a human or animal body to lower the incidences of gastric events in persons using non-steroidal, anti-inflammatory medications, the compound including clinoptilolite.

According to another aspect of the invention, due to its slow release process, cliniptilolite can be employed in a method of treating a human or animal body, which includes using clinoptilolite as an adjuvant to inhibit/manipulate the growth of cancer cells. It gives chemotherapy patients a better quality of life by reducing the side effect normally associated with such treatment.

According to another aspect of the invention, there is provided a use of a medicament for treating a human or animal body to inhibit the growth of cancer cells, the medicament including clinoptilolite.

According to another aspect of the invention, there is provided a compound for treating a human or animal body to inhibit the growth of cancer cells, the compound including clinoptilolite.

According to yet another aspect of the invention, there is provided a wound dressing, which includes an absorbent comprising potentiated clinoptilolite produced according to a method as described.

The term “potentiated clinoptilolite” is meant to indicate clinoptilolite produced using the method of the invention, as described hereinbefore.

The invention will now be described in more detail with reference to the following non-limiting examples and drawings.

### DRAWINGS

In the drawings:

FIG. 1 shows a patient's mean blood alcohol concentration;

FIG. 2 shows a patient's exhaled alcohol concentration;

FIG. 3 shows a patient's mean GIT domain scores;

FIG. 4 shows a patient's mean CNS scores;

FIG. 5 shows a patient's mean blood alcohol concentration;

FIG. 6 shows a patient's exhaled alcohol concentration;

FIG. 7 shows a patient's mean GIT domain scores;

FIG. 8 shows a patient's mean CNS scores;

FIG. 9 shows a patient's mean total pain scores;

FIG. 10 shows a patient's mean number of heartburn episodes;

FIG. 11 shows a patient's mean discomfort scores;

FIG. 12 shows a patient's mean symptom free days;

FIG. 13 shows a patient's mean discomfort scores;

FIG. 14 shows a patient's mean number of heartburn episodes;

FIG. 15 shows a patient's mean gastritis ratings;

FIG. 16 shows a patient's mean erosion ratings;

FIG. 17 shows a normal stomach lining;

FIG. 18 shows a stomach lining subjected to severe gastritis;

FIG. 19 shows a stomach lining of a patient who received placebo during the study period;

FIG. 20 shows mean mercury levels of smoking volunteers;

FIG. 21 shows mean cadmium levels of smoking volunteers;

FIG. 22 shows normalized mercury levels of smoking volunteers;

FIG. 23 shows normalized cadmium levels of smoking volunteers;

FIG. 24 shows a proportion of participants responding to treatment after a number of weeks;

FIG. 25 shows an IBS severity score after a number of periods;

FIG. 26 shows mean SD total severity scores after a number of periods;

FIG. 27 shows severity of pain scores after a number of periods;

FIG. 28 shows mean SD pain severity scores after a number of periods;

FIG. 29 shows a mean number of days with pain before treatment;

FIG. 30 shows a mean SD number with pain on a number of days;

FIG. 31 shows a mean severity distension scores before treatment;

FIG. 32 shows a mean SD severity of distension scores on a number of days;

FIG. 33 shows mean bowel habit satisfactory scores after a number of treatment days;

FIG. 34 shows a mean SD bowel habit satisfaction scores after a number of treatment days;

FIG. 35 shows a mean interference with life scores after a number of treatment days; and

FIG. 36 shows a mean SD interference with life scores after a number of treatment days.

### DETAILED DESCRIPTION OF THE INVENTION

### Example 1A

It has been shown that oral administration of potentiated clinoptilolite reduces the effects of alcohol overintoxication.

Just under half of adult men (45 percent) and one-fifth of women (17 percent) currently consume alcohol. For the total population, the rate is 28 percent, which translates to 8.3 million South Africans, 15 years or older, who currently consume alcohol.

Ethanol in the blood affects the brain and other tissues until it is metabolized by the liver through oxidation, detoxifying the substance and rendering it harmless to tissues and organs. A small amount of the ethanol is excreted via the lungs and urine. There are numerous anecdotal claims for substances that are able to either reduce alcohol intoxication or improve the symptoms of over indulgence. None were ever proven in randomised clinical trials.

The potenitaled clinoptilolite falls under a family of natural and/or synthetic hydrated aluminosilicates. These, are molecules with a well-defined, microporous, crystalline structure which carries an excessive negative charge, usually compensated for by cations. Void spaces within the frameworks, 3 to 10 A in diameter, are capable of hosting cations, water, or other organic molecules that in turn can be selectively exchanged for other species. The porous structure allows hydrated aluminosilicates to have a unique “molecular sieving” ability. The aim of this pilot study was to evaluate the purported effects of potenitaled clinoptilolite on blood and breath alcohol levels with and without food and to establish any effect on the so-called hangover symptoms.

Potenitaled clinoptilolite was evaluated in 2 separate double blind, randomised, placebo controlled cross over studies. Each trial included 2 groups consisting of 5 subjects per group, amounting to a total of 10 subjects and 20 data point assessments (subjects served as their own controls). Volunteers were administered oral 6 g potenitaled clinoptilolite 2.4D as follows:

(a) On empty stomach with 75 ml alcohol (blood alcohol levels).
(b) After a meal and an evening of “free” drinking and eating breath alcohol as well as Central

Nervous System (CNS) and Gastro-intestinal Tract (GIT) symptoms were recorded. A participant received what ever drink he/she preferred, receiving an average of at least 2 drinks (i.e. 2×1 tot equivalent to 10 ml alcohol) every hour over a 3 hour period, after which potenitaled clinoptilolite 2.4D (6 g) was administered. All alcohol and time of consumption was documented and repeated in exactly the same manner when individual returned for the cross over phase. The washout period was 72 hours.

Persons documented all food taken during the day of the first leg of the phase in order to repeat his/her intake in the second leg. Persons received a meal during the study of his/her choice, which too was documented and repeated in second leg.

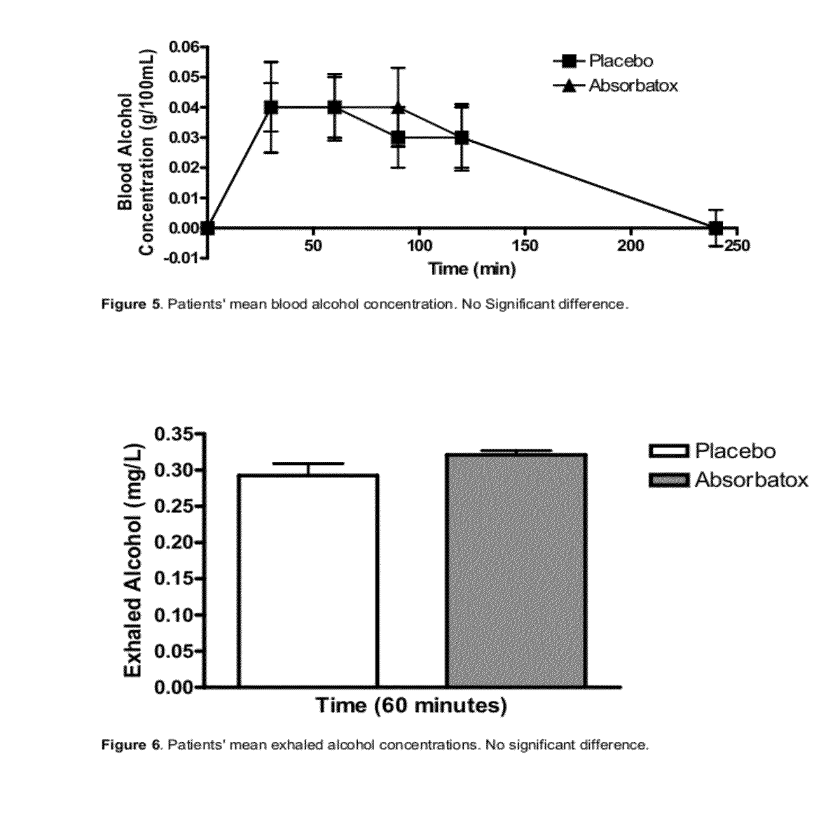

Pertaining to the blood alcohol levels, potenitaled clinoptilolite had no effect on blood (FIG. 5) or breath alcohol levels (FIG. 6) and there was also no change in alcohol absorption with or without food.

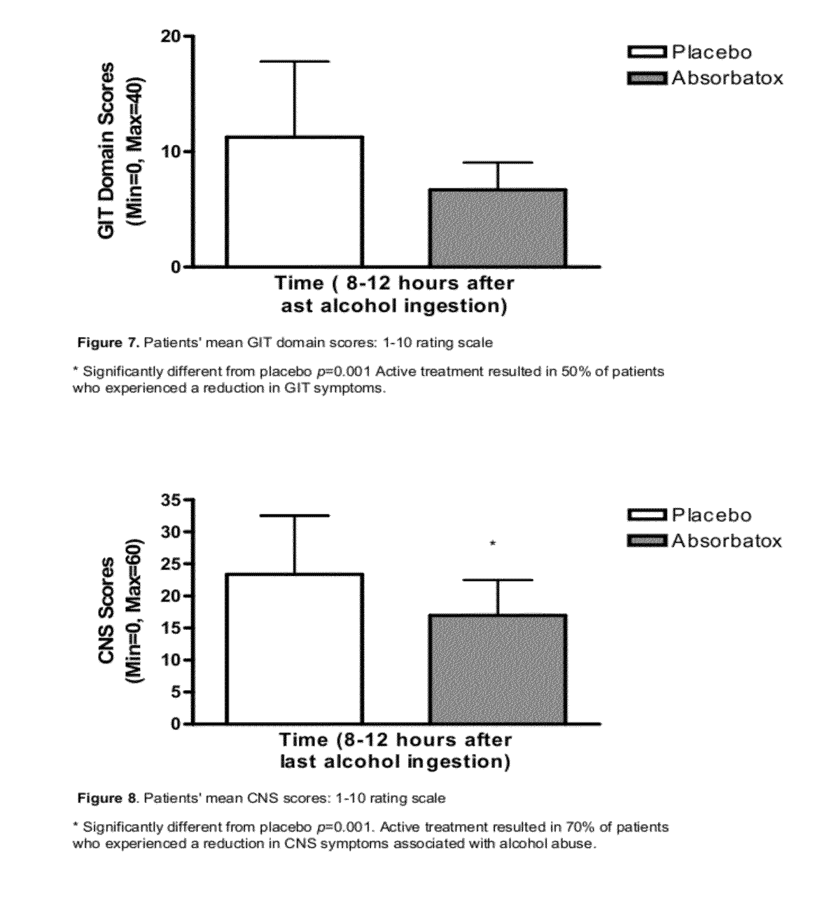

In the second study with statistical analysis, using a Wilcoxon Signed Rank Test, a significant decrease of these symptoms was observed when comparing the treated vs the placebo groups respectively. potenitaled clinoptilolite significantly (p=0.001) improved both GIT and CNS symptoms. With respect to the questionnaire the 10 questions were divided into two groups, namely the Cognitive and GIT Domains respectively. The Cognitive Domain contained questions pertaining to fatigue, irritability, alertness, light sensitivity, sound sensitivity and headache. GIT Domain contained questions pertaining to thirst, nausea, vomiting and bowel irritation. It was also noteworthy that none of the participants experienced nausea after using the potenitaled clinoptilolite, compared to the 40% that did experience nausea in the placebo group. Fifty percent of patients experienced an overall reduction in GIT symptoms (p=0.001) (FIG. 7) whereas 70 percent of patients reported a reduction in the CNS symptoms (p=0.001) (FIG. 8) of hangover.

Alcohol overindulgence is associated with gastrointestinal disturbances such as upper abdominal pain, nausea and vomiting, and CNS symptoms such including headache, irritability, dizziness etc. These GIT symptoms manifest possibly due to direct irritation of the stomach lining (gastritis), fatty liver, increased gastric acid and pepsin secretion, and pancreatic and other intestinal secretions. Headaches associated with hangovers have been attributed in some part due to the effect of alcohol on neurotransmitters and hormones such as histamine, serotonin and prostaglandins, which have been implicated in the pathogenesis of headaches.

This study indicated that potenitaled clinoptilolite does not have an effect on blood or breath alcohol levels. The results obtained from the second study demonstrate an overall significant reduction in CNS and GIT hangover symptoms. The exact mechanism of action is not elucidated in this study, but the reported effects on heartburn and gastritis may indicate that potenitaled clinoptilolite possibly binds the offending H⁺ ions, pepsin, and biological active amines and nitrates. Further evaluation is warranted by the outcomes of these studies. potenitaled clinoptilolite is a non-absorbable substance with potential benefit in preventing and reducing hangover symptoms.

### Example 1B

Just under half of adult men (45 percent) and one-fifth of women (17 percent) currently consume alcohol. For the total population, the rate is 28 percent, which translates to 8.3 million South Africans, 15 years or older, who currently consume alcohol.

Ethanol in the blood affects the brain and other tissues until it is metabolized by the liver through oxidation, detoxifying the substance and rendering it harmless to tissues and organs. A small amount of the ethanol is excreted via the lungs and urine. There are numerous anecdotal claims for substances that are able to either reduce alcohol intoxication or improve the symptoms of over indulgence. None were ever proven in randomised clinical trials.

Potenitaled clinoptilolite falls under a family of natural and/or synthetic hydrated aluminosilicates. These, are molecules with a well-defined, microporous, crystalline structure which carries an excessive negative charge, usually compensated for by cations. Void spaces within the frameworks, 3 to 10 Å in diameter, are capable of hosting cations, water, or other organic molecules that in turn can be selectively exchanged for other species. The porous structure allows hydrated aluminosilicates to have a unique “molecular sieving” ability.

The aim of this pilot study was to evaluate the purported effects of Absorbatox® on blood and breath alcohol levels with and without food and to establish any effect on the so-called hangover symptoms.

Potenitaled clinoptilolite was evaluated in 2 separate double blind, randomised, placebo controlled cross over studies. Each trial included 2 groups consisting of 5 subjects per group, amounting to a total of 10 subjects and 20 data point assessments (subjects served as their own controls). Volunteers were administered oral 6 g Absorbatox® 2.4D as follows:
(c) On empty stomach with 75 ml alcohol (blood alcohol levels).
(d) After a meal and an evening of “free” drinking and eating breath alcohol as well as Central Nervous System (CNS) and Gastro-intestinal Tract (GIT) symptoms were recorded.

A participant received what ever drink he/she preferred, receiving an average of at least 2 drinks (i.e. 2×1 tot equivalent to 10 ml alcohol) every hour over a 3 hour period, after which Absorbatox® 2.4D (6 g) was administered. All alcohol and time of consumption was documented and repeated in exactly the same manner when individual returned for the cross over phase. The washout period was 72 hours.

Persons documented all food taken during the day of the first leg of the phase in order to repeat his/her intake in the second leg. Persons received a meal during the study of his/her choice, which too was documented and repeated in second leg.

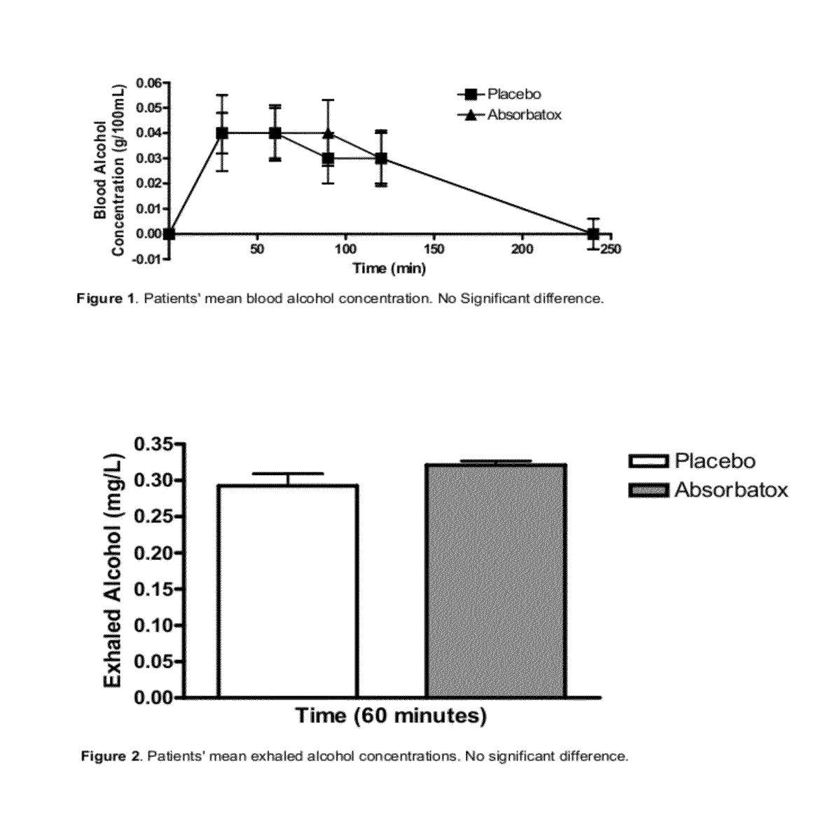

Pertaining to the blood alcohol levels, potenitaled clinoptilolite had no effect on blood (FIG. 1) or breath alcohol levels (FIG. 2) and there was also no change in alcohol absorption with or without food.

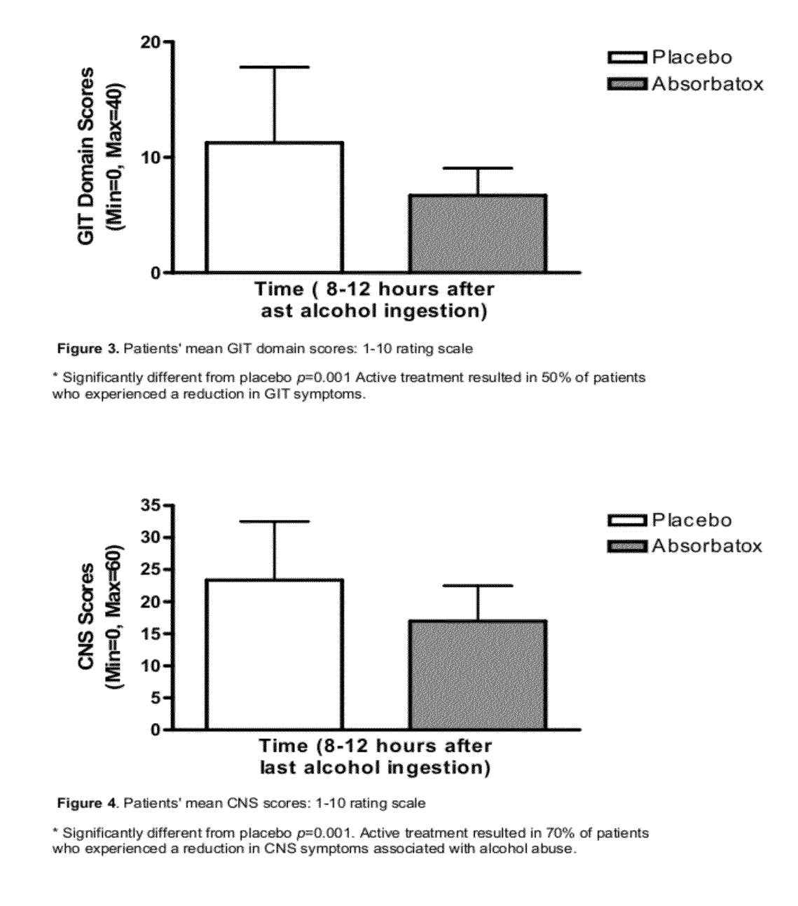

In the second study with statistical analysis, using a Wilcoxon Signed Rank Test, a significant decrease of these symptoms was observed when comparing the treated vs the placebo groups respectively. Absorbatox® significantly (p=0.001) improved both GIT and CNS symptoms. With respect to the questionnaire the 10 questions were divided into two groups, namely the Cognitive and GIT Domains respectively. The Cognitive Domain contained questions pertaining to fatigue, irritability, alertness, light sensitivity, sound sensitivity and headache. GIT Domain contained questions pertaining to thirst, nausea, vomiting and bowel irritation. It was also noteworthy that none of the participants experienced nausea after using the potenitaled clinoptilolite, compared to the 40% that did experience nausea in the placebo group. Fifty percent of patients experienced an overall reduction in GIT symptoms (p=0.001) (FIG. 3) whereas 70 percent of patients reported a reduction in the CNS symptoms (p=0.001) (FIG. 4) of hangover.

Alcohol overindulgence is associated with gastrointestinal disturbances such as upper abdominal pain, nausea and vomiting, and CNS symptoms such including headache, irritability, dizziness etc. These GIT symptoms manifest possibly due to direct irritation of the stomach lining (gastritis), fatty liver, increased gastric acid and pepsin secretion, and pancreatic and other intestinal secretions. Headaches associated with hangovers have been attributed in some part due to the effect of alcohol on neurotransmitters and hormones such as histamine, serotonin and prostaglandins, which have been implicated in the pathogenesis of headaches.

This study indicated that potenitaled clinoptilolite does not have an effect on blood or breath alcohol levels. The results obtained from the second study demonstrate an overall significant reduction in CNS and GIT hangover symptoms. The exact mechanism of action is not elucidated in this study, but the reported effects on heartburn and gastritis may indicate that potenitaled clinoptilolite possibly binds the offending H⁺ ions, pepsin, and biological active amines and nitrates. Further evaluation is warranted by the outcomes of these studies. potenitaled clinoptilolite is a non-absorbable substance with potential benefit in preventing and reducing hangover symptoms.

### Example 2

It has been shown that oral administration of potentiated clinoptilolite reduces GORD associated clinical symptoms and protects against NSAID induces gastritis.

Heartburn is a debilitating disease that not only influences the quality of life of patients but also has a great impact on health system costs. It has been shown that up to 81% of patients suffering from heartburn would be diagnosed positively with gastro oesophageal reflux disease (GORD). Patients suffering from GORD can either be classified as being endoscopically negative or positive after upper gastro-intestinal endoscopic evaluation Patients with endoscopically negative gastro oesophageal reflux disease (ENGORD) experience heartburn symptom severity and decrements in their quality of life comparable to patients with erosive oesophagitis. Patients suffering from occasional heartburn usually self-medicate with antacids for symptomatic relief. The aim of therapy is to improve quality of life of patients suffering from heartburn manifesting from GORD. Treatment regimens usually depend on the severity and intensity of symptoms⁴. The use of proton pump inhibitors (PPI's) has become the gold standard in practice with the majority of patients responding well to treatment (i.e. with or without erosive oesophagitis). The “treat as needed” approach with PPI's is suitable for younger patients who suffer from non-erosive oesophagitis or Grade A and B oesophagitis. The latter patients may not experience heartburn on a daily basis and require a PPI only when experiencing episodes of heartburn.

PPI's also reduce ulcer formation in patients using non-steroidal anti-inflammatory drugs (NSAIDs) but may often result in drug-drug interactions especially in patients taking multiple agents for co-morbid conditions. There is therefore a need for safer, affordable and more accessible products able to protect at-risk patients against the side effects of GORD and NSAIDs without drug-drug interactions.

The main benefit of non-selective NSAIDs derives from their anti-inflammatory and analgesic effects. Patients who take NSAIDs may develop serious gastrointestinal (GI) side effects in both the upper and lower GI tract. It has been determined that NSAIDs are ulcerogenic, with a greater risk for gastric ulcers than duodenal ulcers⁵. In many Western societies, NSAID use may be responsible for more ulcer diseases than H. pylori. Low-dose NSAID use, even cardioprotective doses of aspirin, can also be ulcerogenic.

Introduction of COX-2 specific inhibitors have reduced the risk of ulcers by 50%. This however is offset by the increased risk of adverse cardiovascular outcomes⁶, limiting their use in patients with co-morbid conditions such as coronary artery disease. It is often these older higher risk (cardiovascular risk) patients who also suffer from chronic musculoskeletal pains such as osteoarthritis and needs constant or at least occasional NSAID therapy for pain relief. Safer, affordable and accessible products are thus needed for protection against the side effects of NSAIDs without drug-drug interactions. NSAID induced ulcer are also used to evaluate the gastro-protective properties of other drugs such as PPI's.

The many uses of hydrated aluminosilicate crystals in both industry and agriculture are attributable to their three-dimensional structure, which endows them with specific physicochemical properties including: ion exchange capacity, adsorbent nature, size exclusion frame work as well as catalytic properties. Additionally, they have been claimed to exhibit diverse biological activities, which may include uses such as adjuvant therapy to anticancer therapy, adjuvant for vaccines, treatment of heartburn and as anti diarrhoeal agents. Various studies have shown that hydrated aluminosilicates have no toxic effects both in humans and in animals

Potenitaled clinoptilolite is a synthetically enhanced hydrated aluminosilicate that falls under the zeolite family. Potenitaled clinoptilolite is a unique aluminosilicate crystal with properties differentiating it from other aluminosilicates, due to its patented process resulting in a higher cation exchange capacity (CEC) and specific size exclusion frame (through manipulating the pore size). These Absorbatox® molecules have a well-defined, microporous, crystalline structure which carries an excessive negative charge, usually compensated for by cations. Void spaces within the frameworks, 3 to 10 Å in diameter, are capable of hosting cations, water, or other organic molecules, which can then be selectively exchanged for other species. The result is a designer product able to exchange specific types of molecules. It may also bind biologically active amines. It is an ion exchange agent and this makes it a possible agent to reduce gastric pH and symptomatic relief of heartburn. Hydrated aluminosilicates are also known to adsorb toxins from pathogens such as fungi or bacteria which may cause abdominal discomfort, and decreases acidity. Antibacterial and antiviral properties of these hydrated aluminosilicates have also been reported.

Therefore the aim of this study on potenitaled clinoptilolite was to (a) establish its effect in patients with heartburn (its ability to bind hydrogen ions and nitrates may benefit patients with GORD by reducing acidity and the effect of gastric enzymes) and (b) to evaluate its gastro-protective ability in patients with NSAID induced gastritis.

### ENGORD Study

A double blind, placebo controlled trial, with randomization to trial treatment into one of the two treatment arms was performed. Volunteers were randomized to receive either a placebo or potenitaled clinoptilolite 2.4D (C35) (750 mg capsule) three times daily for a period of 14 days. Volunteers were assessed on use of rescue medication, symptom free days, discomfort, and pain by means of questionnaires to be documented in a patient diary. Rescue medication, PPI, (Nexium®, esomeprazole 20 mg) was part of on demand therapy for all the patients.

Volunteers over the age of 18 years old who after endoscopy, were diagnosed with ENGORD, were recruited on to the trail after signing informed consent. The study enrolled 25 patients. Thirteen patients were randomized to receive potenitaled clinoptilolite and twelve to receive placebo.

### NSAID Induced Gastritis Study

In this 14 day double blind placebo controlled pilot study, 22 healthy volunteers were enrolled. Volunteers were randomized to receive orally either 1500 mg 2.4D (C35) or placebo three times daily, plus 500 mg naproxen twice daily. Naproxen has been used as comparator NSAID in evaluating the clinical benefits of COX-2 inhibitors such as rofecoxib and meloxicam. NSAIDs such as naproxen are known to cause non-symptomatic mucosal breaks in almost 80% of patients. These asymptomatic ulcers are significantly reduced by the COX-2 inhibitors, hence the selection of naproxen as offending drug (NSAID causing ulceration) in this pilot study.

All volunteers were over the age of 18 years, and underwent gastroscopic evaluation of their stomach linings prior to and on day 14 of the study to evaluate gastric events and the status of the gastric mucosa. Volunteers also filled in a daily symptom diary.

Symptomatic differences between the groups receiving placebo and potenitaled clinoptilolite were analyzed by means of an unpaired t-test and Mann-Whitney test, at 95% confidence interval. Differences in occurrence of gastritis and erosions were analyzed by means of a Wilcoxon signed-rank test, at 95% confidence interval. Data was considered significant at p≦0.05. The software program GRAPHPAD was used for data analysis.

### ENGORD Study

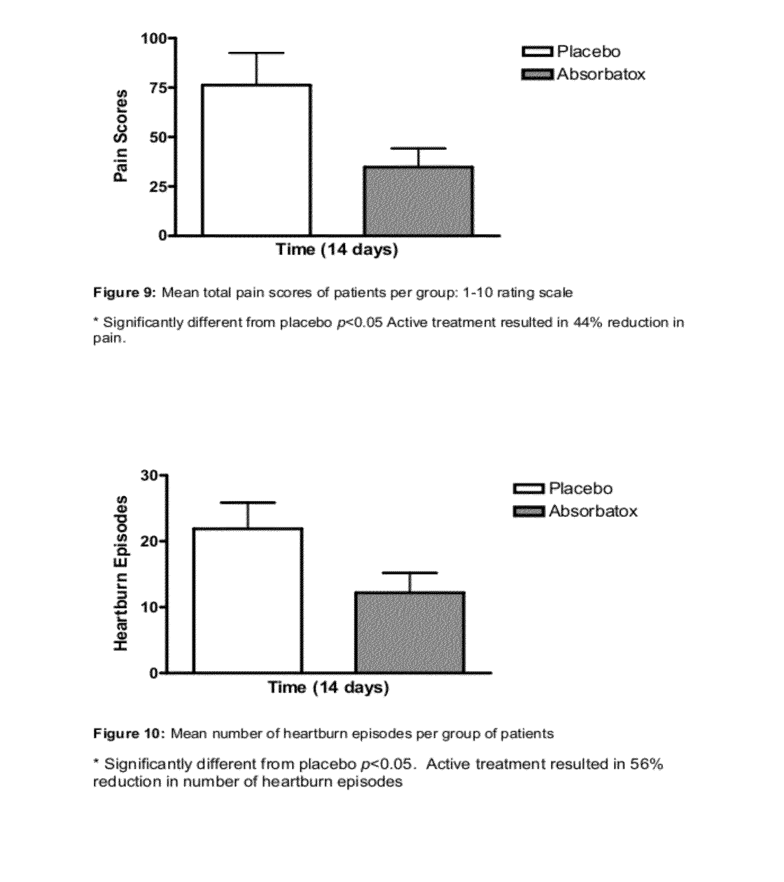

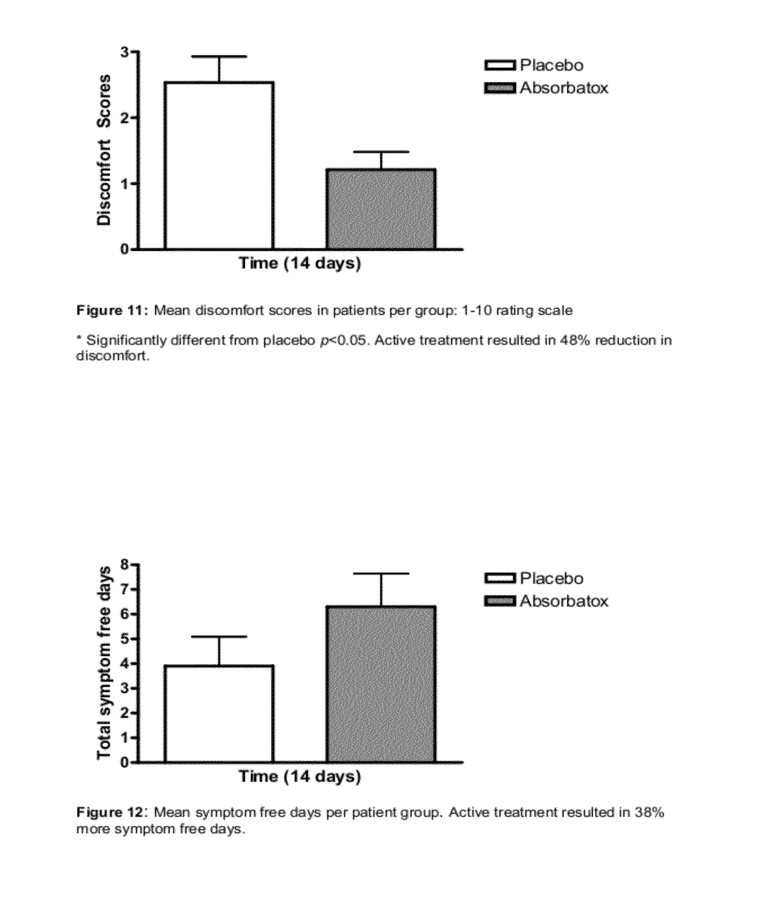

Potenitaled clinoptilolite significantly reduced the pain (44% reduction), discomfort (48% reduction), and number of heartburn episodes (56% reduction) in patients with GORD (p<0.05) (FIG. 9-11). The potenitaled clinoptilolite group also experienced more symptom free days (i.e. 29% vs. 42%) compared to the placebo treated group (FIG. 12). Adverse events are reported in Table 1. A total of five patients did not complete the study successfully; three patients did not return on their scheduled visits (2 received placebo, 1 Absorbatox).

TABLE 1
Table 1: Indicating side effects reported
by volunteers during both studies

| | Absorbatox (n = 10) | Placebo (n = 10) |
|---|---|---|
| Constipation | 2/10 | 0/10 |
| Bloatedness | 3/10 * | 0/10 |
| Nausea | 1/10 | 1/10 |
| Sleeplessness | 1/10 | 0/10 |
| No side- effects | 4/10 | 9/10 |

### NSAID Induced Gastritis Study

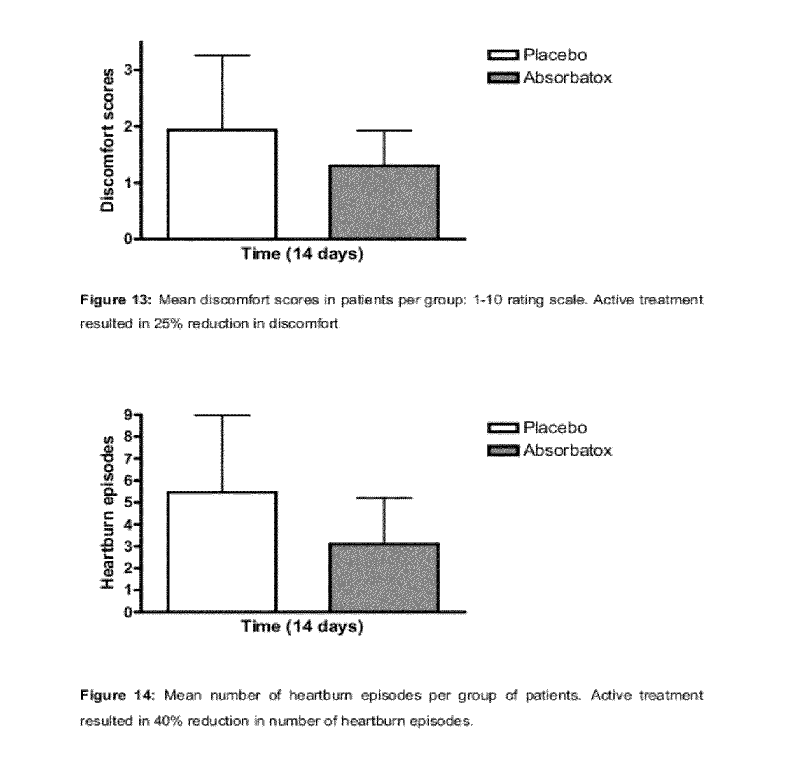

Potentiated clinoptilolite resulted in a reduction of clinical symptoms (25% reduction in discomfort and 40% reduction in heartburn) (FIG. 13,14). The use of rescue medication in the group was potenitaled clinoptilolite slightly lower than in the group receiving the placebo however this was not significant.

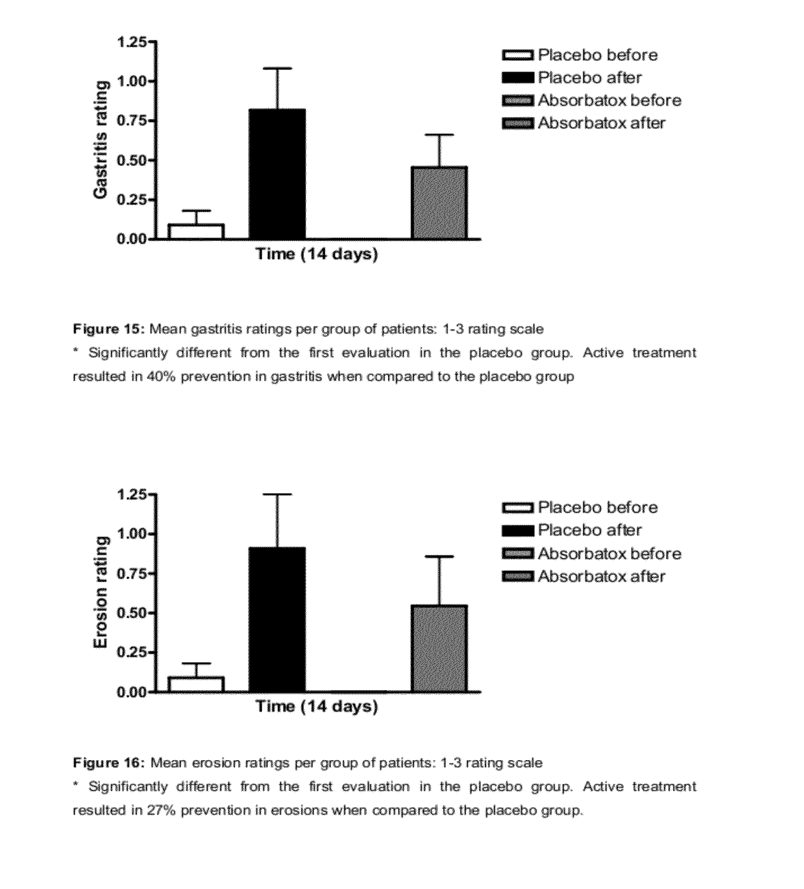

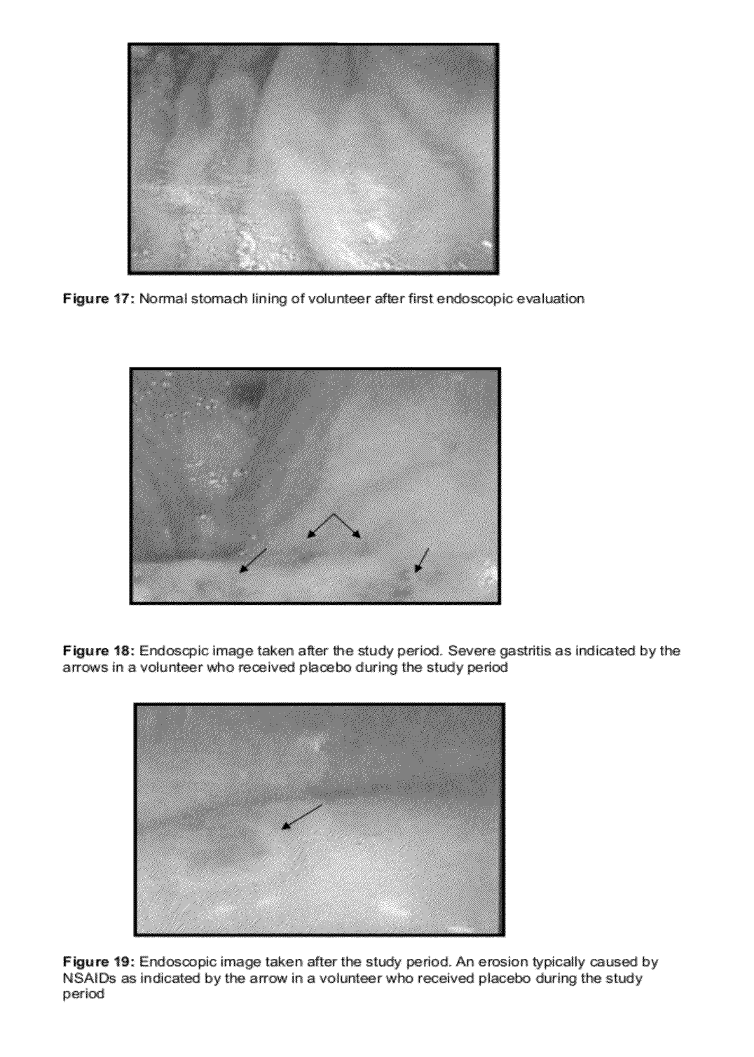

Pertaining to gastric events, gastritis and erosions of the stomach lining were evaluated via endoscopy for each volunteer and graded according to a 1-3 rating scale. Evaluations were done prior to and after completion of the study period. A significant increase in the occurrence of gastritis and erosions were observed in volunteers assigned to the placebo treatment arm (p<0.05) (FIG. 15-19). No significant changes in occurrence of gastritis or erosions were seen when comparing before and after evaluations for the group that received potentiated clinoptilolite (FIG. 15, 16). potentiated clinoptilolite is thus associated with a prevention in gastritis (40%) and incidences of erosions (27%). Atrophy occurred in one volunteer receiving the placebo. One volunteer that received potentiated clinoptilolite experienced slight flatulence, and one volunteer in the placebo group complained of bloatedness.

The first study indicates the potential for use of potentiated clinoptilolite in the treatment of non-complicated GORD. Results obtained from this study demonstrate overall clinical improvement, up to 50%, in several of the study outcome groups. The exact mechanism of action of potentiated clinoptilolite was not elucidated in this study but may be ascribed to hydrogen ion trapping.

The main outcome in the second study was achieved in that potentiated clinoptilolite reduced gastric events and symptoms induced by naproxen. potentiated clinoptilolite resulted in fewer events of gastritis and also a reduction of erosion and atrophy. Heartburn and discomfort symptom reduction echoed the findings of the ENGORD study.

It is possible that the effect is due to simple binding of the offending H⁺ ions and pepsin on the gastric mucosa. It is also possible that potentiated clinoptilolite binds to biological active substances resulting in fewer episodes of gastritis and limiting the extent of collateral tissue damage.

It has further been suggested that intraluminal and enterobacteria bacteria play a significant role in the pathogenesis and lesion formation of gastro-intestinal damage induced by NSAID's^{8,9}.

Antibacterial and antiviral properties of potentiated clinoptilolite may also be responsible for the reduction of gastric events and clinical.

In these small studies this magnitude of effect was already big and therefore warrants evaluation in a phase III study. Potentiated clinoptilolite is a non-absorbable substance with potential benefit in reducing GORD associated clinical symptoms and protecting against NSAID induced ulcers. Potentiated clinoptilolite is effective by possible binding of the offending H⁺ ions and binding to biologically active substances.

### Example 3

In a double blind randomized, placebo controlled study it was shown that oral administration of potentiated clinoptiline has been proven to lower high levels of heavy metals in humans.

These graphs represent the results of participants that completed the study after 2 months: Placebo=7 volunteers, potentiated clinoptilolite=11 volunteers

The heavy metal results of 6 patients are still outstanding.

The quality of life questionnaire results are outstanding for all the patients.
All volunteers were smokers (±15 cigarettes per day)
Blood was drawn before commencement of the study, and after 2 months.
Absorbatox was taken as follows: 1 capsule (750 mg) twice daily

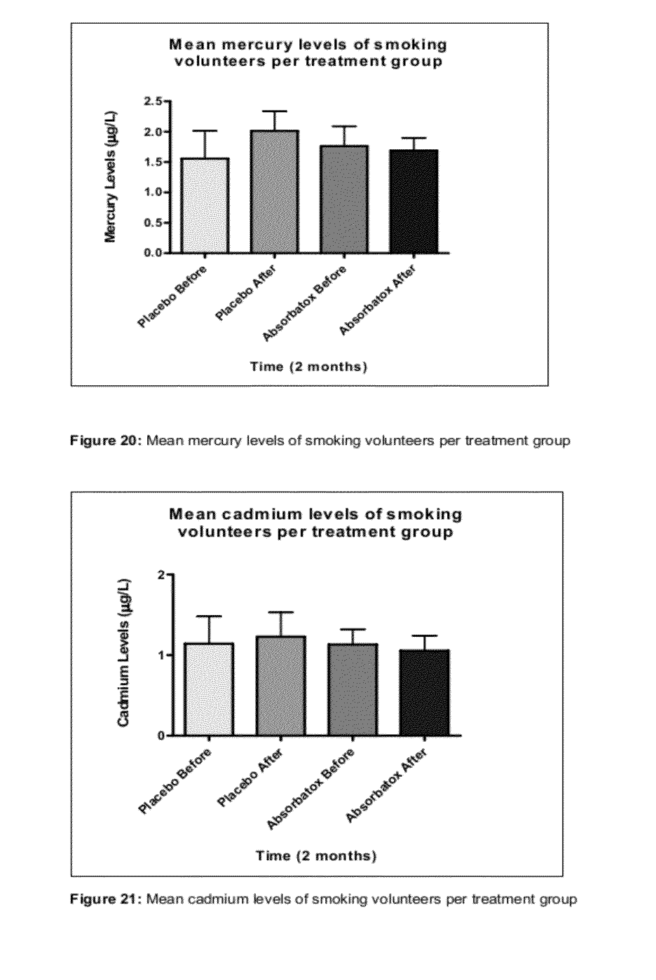

FIG. 20, 21: The first two graphs represent the mean heavy metal blood levels of the volunteers per treatment group.

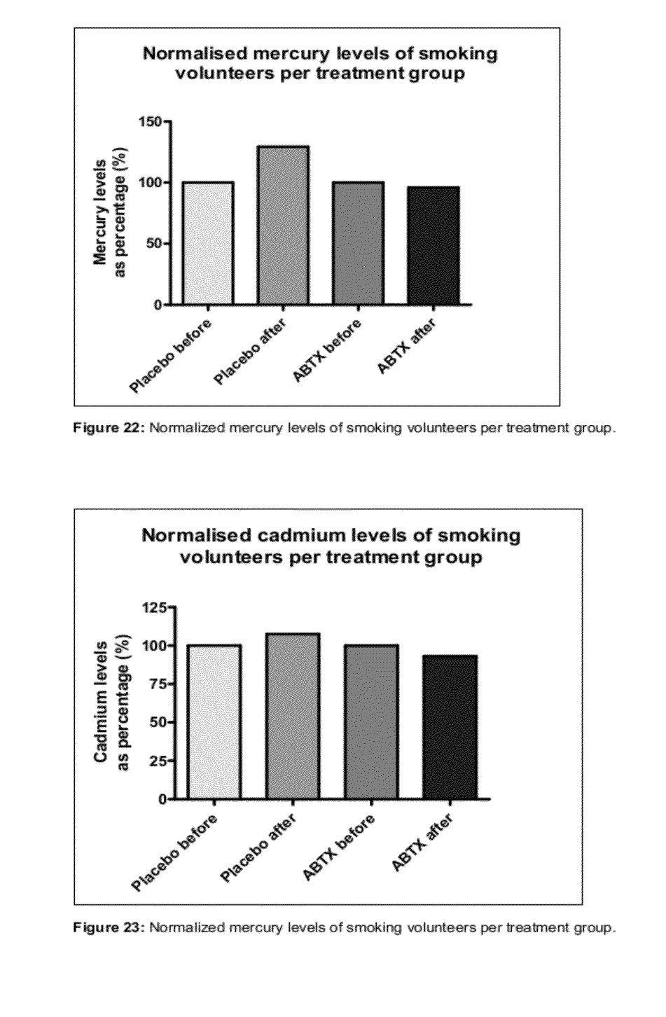

FIG. 22, 23: The last two graphs represent the values of the heavy metal blood levels as percentages in order to obtain normalized “before” levels in order to compare the “after” levels with each other. According to these preliminary results, potentiated clinoptilolite resulted in a 26% prevention of Hg (mercury) blood level elevation and a 13% prevention of Cd (cadmium) blood level elevation. I have not yet confirmed statistical significance.

Irritable Bowel Syndrome (IBS) is one of the most common gastrointestinal disorders encountered by primary care physicians and gastroenterologists. It is a chronic disorder characterised by abdominal discomfort, bloating and altered defecation patterns. It is well known that IBS accounts for great health care resource utilisation and health care costs in general. A poor quality of life, including physical and psychological levels of wellbeing, work absenteeism, and various direct and indirect financial burdens are well associated with IBS. Although the pathophysiology is better understood than in the past, much is yet to be elucidated. As a result IBS management has often led to poor treatment outcomes. Current pharmacological treatments are either aimed at the predominant symptom (end-organ) and/or relieving an associated affective disorder. Partly from a lack of conventional western medicine, several sufferers have turned to complimentary medicine. It is estimated that approximately 40% of IBS suffers seek symptom relief from alternative and complimentary medication. These medical therapies and practices have included hypnotherapy, forms of herbal therapy and certain probiotics.

In the current trial potentiated clinoptilolite as produced in terms of the method of this invention called “C35” was used and tested as a new CAM agent in IBS. This is the first clinical study in which the natural zeolite is assessed for efficacy in IBS patients. Due its unique structure, ion and gas adsorbing properties and beneficial effects on the GI tract, potentiated clinoptilolite “C35” was worthy of investigation in a randomised, double-blind, placebo controlled trial in IBS patients.

Since IBS is a chronic disorder, safety and tolerability are valid factors to consider when a certain drug is used for one or more of many symptoms, as sufferers are likely to consume medication over long periods of time. Potentiated clinoptilolite “C35” has not shown side effects in various animal and human studies, and have lacked significant side effects in the present trial. Potentiated clinoptilolite “C35” might be effective in the management of IBS in patients with similar characteristics as this trial. Although largely unavoidable, the placebo effect and the small sample size may be labelled as study limitations.

The American College of Gastroenterology (ACG) has developed a grading tool in which the quality of trial methodology is evaluated and quantitatively scored on a 14 point scale. The Quantitative Assessment of Trial Methodology Scale (QATMS) uses 14 different criteria. For each separate criterion a value of one is awarded if the specific trial met the requirement stated in the criterion. Therefore, a maximum of 14 can be scored, and quality score of >10 is considered as “high quality”, quality score between 6 and 10 as “intermediate”, while a trial scoring lower than 5 is labelled as low quality trial methodology. The criteria are as follows:
1 Rome criteria used to identify patients with IBS;
2 randomised;
3 parallel-trial design (no cross-over);
4 double-blinded;
5 account of patient disposition (discontinuations, withdrawals, etc.);
6 no placebo run-in;
7 baseline assessment of symptoms;
8 minimum treatment duration of 8-12 weeks;
9 follow up of symptoms after treatment is stopped;
10 compliance with treatment is measured;
11 sample size calculation is provided and adequate sample size enrolled;
12 primary outcome measure is improvement of global IBS symptoms;
13 primary outcome measure is based on patient assessment; and
14 primary outcome measure uses a validated scale to assess IBS symptoms.

The methodology used in this particular trial was of high quality, as a QATMS score of 11 would be achieved according to the ACG. All criteria is met except, 8 (minimum treatment duration of 8-12 weeks), 9 (follow up of symptoms after treatment is stopped) and 11 (sample size calculation is provided and adequate sample size enrolled).

It should be emphasised that this was a pilot study in which treatment duration of four weeks is considered more than adequate. In fact, previous IBS pilot trials have also used short treatment durations of 4 weeks and even 3 weeks. Furthermore, the published literature has indicated that IBS is characterised by repeated episodes of symptomatic periods lasting 4 weeks on average. Abdominal pain/discomfort and distension occur between 28%-33% of days over 12 week period, lasting an average of five days per episode. In addition, IBS patients were only considered for the current trial if they had at least report two episodes of pain/discomfort per week before the study. A four week treatment was therefore considered valid as patients were likely to experience at least two episodes of discomfort per week. With regards to the small sample of 31 patients (ITT) analysed, it must be noted that the recruitment was a great challenge. However, from the results it was clear that a trend towards improvement, although not significant in all parameters, were observed.

This trial has recruited patients from various resources using inclusion/exclusion criteria in concordance with latest literature recommendations. It must be mentioned that various IBS trials have ruled out organic cause by standard laboratory and radiological tests, and rectosigmoidoscopy. However, due to a tight budget and with the intention to keep this trial a non-invasive one, laboratory and radiological test was not performed on IBS candidates. This may easily be labelled as a limitation of this study, but rigorous diagnostic testing in typical IBS cases has been criticised before, and it is often thought that symptoms should lead the diagnosis rather than extensive and expensive diagnostic testing. In typical IBS patients with no alarm symptoms current best evidence does not support the routine use of blood test, stool studies, breath tests, abdominal imaging or lower endoscopy in order to exclude organic gastrointestinal disease. Alarm symptoms, referred to as red-flags, remains the key indicators of possible diseases other than IBS. Alarm symptoms were incorporated into the Red-flag exclusion sheet as used in the current trial. If an IBS candidate had an alarm symptom, the patient was not considered for participation. Thus, it may be assumed that a full blood count with ESR in conjunction with the use of red-flag exclusion sheet and Rome III symptom based criteria would have effectively exclude patients with gastrointestinal diseases other than IBS. In addition, prevalence of celiac diseases has been found to be higher in a symptom based IBS population compared to the general population. Although extra research is needed, it might be necessary to do a serum screening for tissue transglutaminase in IBS-D patients. The randomisation methodused in this study should have effectively reduced or even neutralised the cases of celiac diseases that might have been missed by the study other studies. An abnormal FBC might also been effective in excluding celiac disease cases.

The potentiated clinoptilolite “C35” group were characterised by an older population than the Placebo group. However, the duration of symptoms, the severity, frequency and impact of symptoms were similar between the two treatment groups. Therefore, all patients had similar disease characteristics before treatment. It could, therefore, be safely assumed that a difference in treatment can be ascribed to a true actual difference and not due to dissimilarities in the degree of symptoms.

Before treatment, both treatment groups were classified as having “moderate” degree of symptoms (Table 6.3), according to the IBS-SSS.

The global assessment question, “In the last 7 days, have you had overall adequate relief of your IBS symptoms?” was used to classify participants as responders or non-responders in the present study. Participants reporting ‘yes” in 50% of treatment weeks (i.e. any two weeks of the 4 week treatment period) were regarded as overall responders.

It is still unclear what constitutes as a responder or a clinical benefit in IBS trials. Recently there have been various consensus reports on the primary and secondary outcomes of IBS trials. It is generally recommended to use a binary (yes/no) measure as a primary endpoint and the FDA has approved this as a primary endpoint in IBS trials.

In the current trial, the binary primary outcome, satisfactory relief (i.e. in the last 7 days have you had satisfactory relief of your IBS symptoms) was first considered in the development of the protocol. Whilst consulting newer literature, it was replaced by the more accepted “Adequate relief” (It must be noted that this amendments has been made before the protocol was submitted for ethical approval).

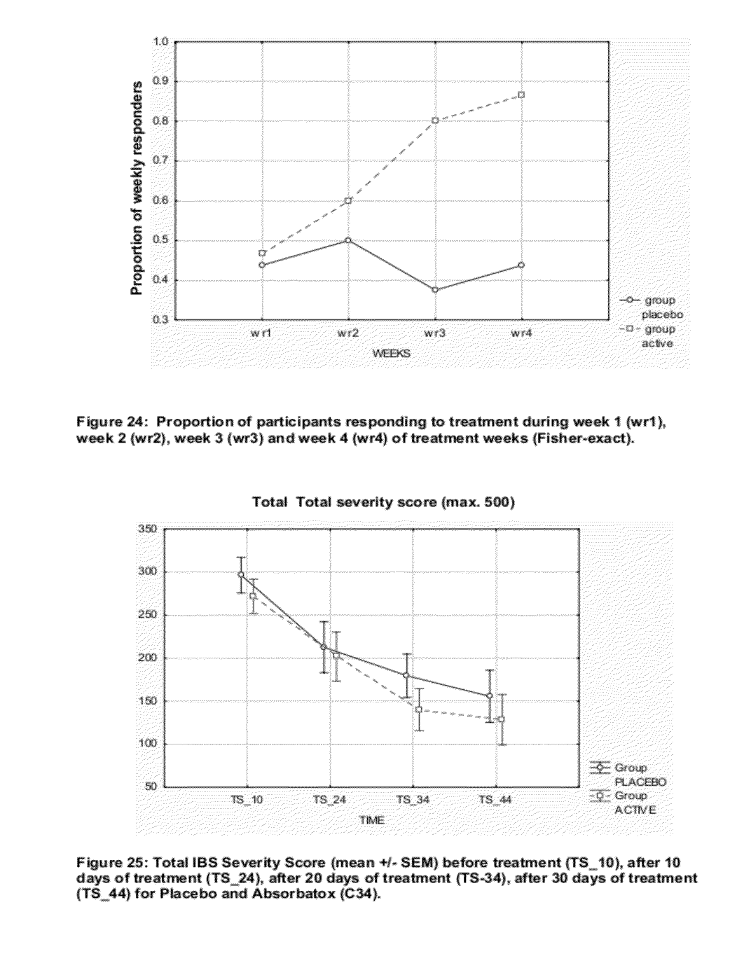

With regards to the findings; the potentiated clinoptilolite “C35” group healed a greater proportion of overall responders than the Placebo group. Although not statistical significant, the improvement trend has favoured the potentiated clinoptilolite “C35” group. A typical IBS trial is one in which a responder rate of 60% compared to a placebo response rate of 45% is achieved. It is unlikely that a statistical significant difference would be reached in such a small sample size as was used in the present trial. In addition, the disorder is characterised by a well known placebo effect during drug trials. Therefore, these results must be viewed in the light of others. It is worth noting that even the best results from large multi-centre studies with approved agents (e.g. tegaserod or alosetron) achieved primary clinical endpoints in less than 70% of patients. Furthermore, it is generally accepted that a 10-15% improvement of the global outcome measure over placebo could be considered as a clinically significant therapeutic gain. In the current trial potentiated clinoptilolite “C35”, according to above mentioned statements, has effectively managed to yield responders of more than 70%, and the effect over Placebo was more than 10-15%. The number of weekly responders (proportion of patients that answered “yes” to adequate relief questionnaire in each week on day 21—after one week of treatment, on day 28—after two weeks of treatment, on day 35—after three weeks of treatment, and on day 42—after four weeks of treatment) were similar at week one and week two, between treatment groups. However, the proportion of weekly responders in the potentiated clinoptilolite “C35” were significantly greater compared to Placebo, after three weeks (FIG. 24). This trend was maintained through week 4 as the proportion of responders in the potentiated clinoptilolite “C35” group was significantly greater than the Placebo group. From these results it was clear that potentiated clinoptilolite “C35” should be used for a minimum period of three weeks before a statistically significant gain over Placebo, in global symptoms, is obtained.

An integrative symptom assessment was done through the use of the IBS-SSS. This questionnaire has been proven to be responsive to treatment effects, and is widely recommended by clinicians and researchers.

Research developers of the IBS-SSS, have stated that a 50 point score change is a reliable indication of symptom improvement. In view of this, a 50 point reduction in symptom scores between baseline (measured on day 10) and at the end of therapy (measured on day 44) was considered a clinical meaningful difference, in this particular trial.

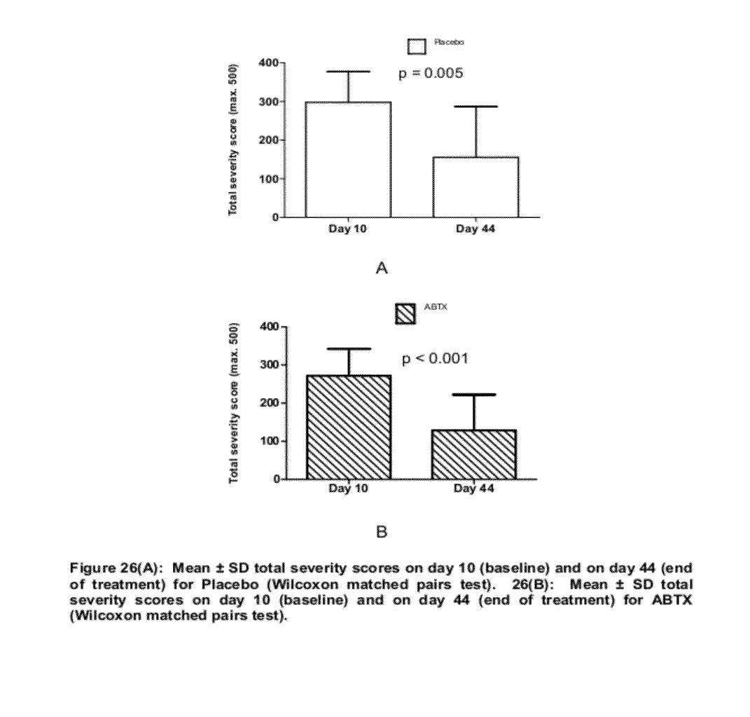

Both the Placebo and the potentiated clinoptilolite “C35” group, showed statistically significant changes across treatment weeks, in the total severity score (FIG. 25, per-protocol analysis (PP)). In the ITT (intention-to-treat) population, the end of treatment total score (day 44) were significantly decreased compared to baseline (day 10) in both the Placebo and potentiated clinoptilolite “C35” group (FIGS. 26(A) & (B)). Finally, both the Placebo and the potentiated clinoptilolite “C35” group had more than 50 point reductions at the end of treatment compared to baseline (Table 6.3). As a matter of fact, a 50% reduction in total score was observed in both groups (day 44 vs. day 10). It was therefore unable to observe treatment differences between the two groups (Table 6.3), as both groups total severity score decreased gradually throughout the treatment weeks. It must be further noted, that both treatment groups changed from having “moderate” symptoms at baseline to “mild” symptoms at the end of treatment, according to IBS-SSS.

In the PP population the Placebo group was not able to show significant reductions in their current pain situation while the potentiated clinoptilolite “C35” group current pain situation decreased significantly across treatment weeks. However, comparisons between the two groups did not reveal any significance on any of the measurement days (day 24, 34, 44; Table 6.4).

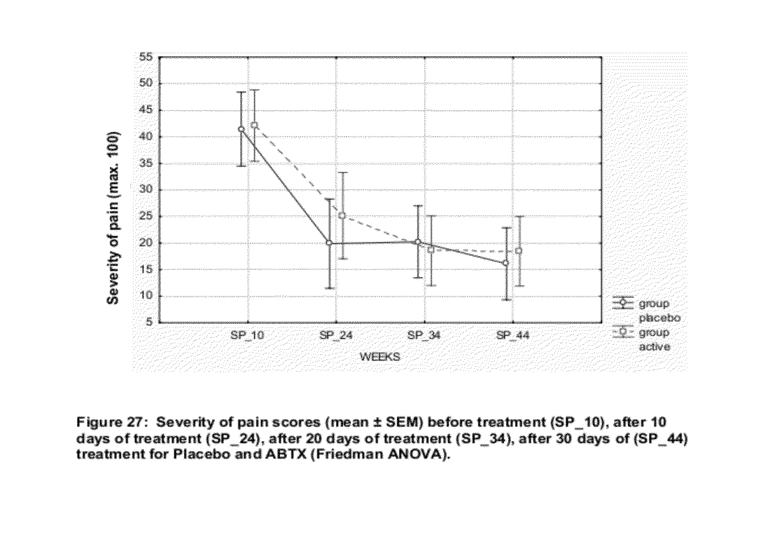

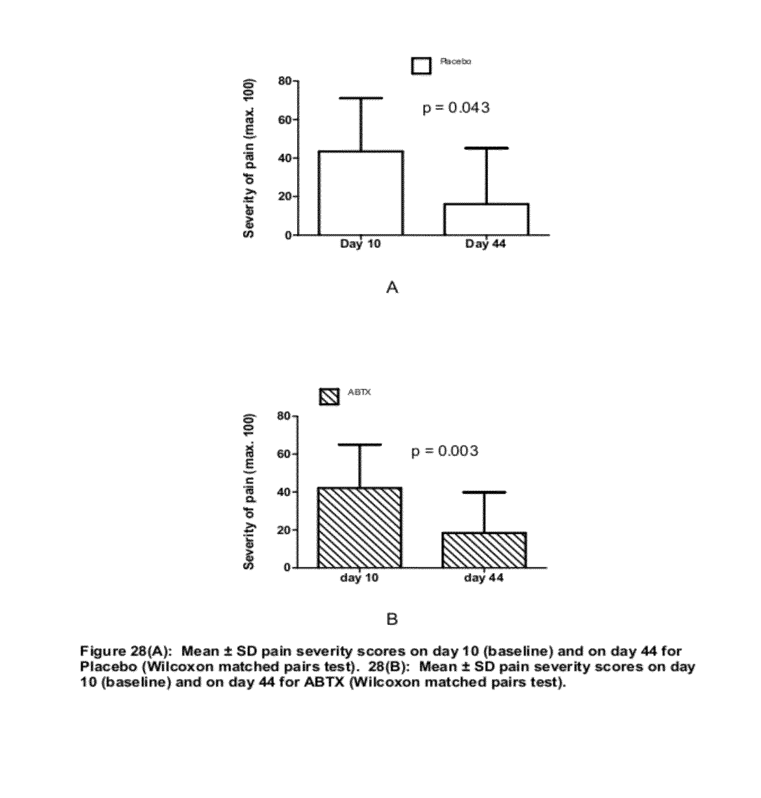

The severity of pain was significantly decreased across treatment weeks in the Placebo as well as in the potentiated clinoptilolite “C35” group (FIG. 27, PP). Likewise, as with “current pain”, no significance was observed in the severity of pain, between treatment groups on any of the measurement days (Table 6.5).

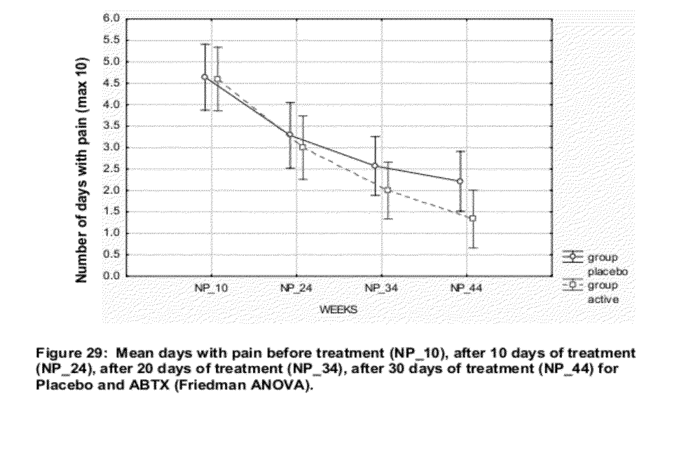

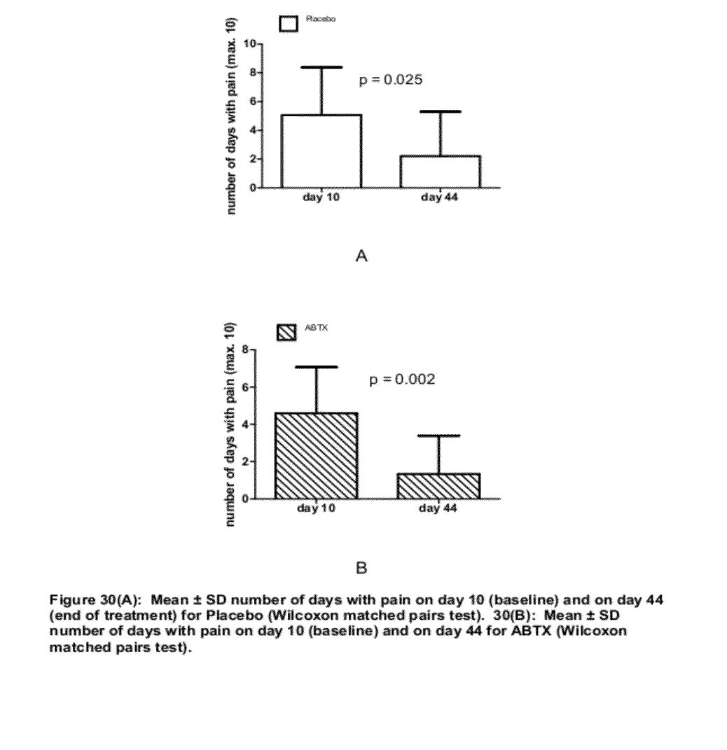

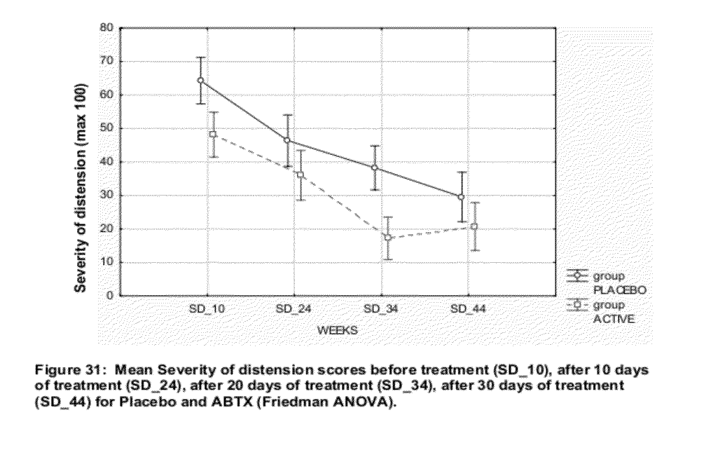

A similar trend was observed in the mean number of days with pain, as both groups showed fewer days with pain across treatment weeks (FIG. 29, PP) and significant changes at the end of treatment compared to baseline (FIG. 30, ITT). Once again, no treatment differences between Placebo and potentiated clinoptilolite “C35” was observed in mean number of days on any of the assessment intervals (Table 6.6).

With regards to distension, a rather similar pattern was observed as with the pain findings; both groups showed significant reduction in “currently having distension” across treatment weeks (PP). During treatment, there was no difference in values between Placebo and potentiated clinoptilolite “C35” on any of the days (Table 6.7).

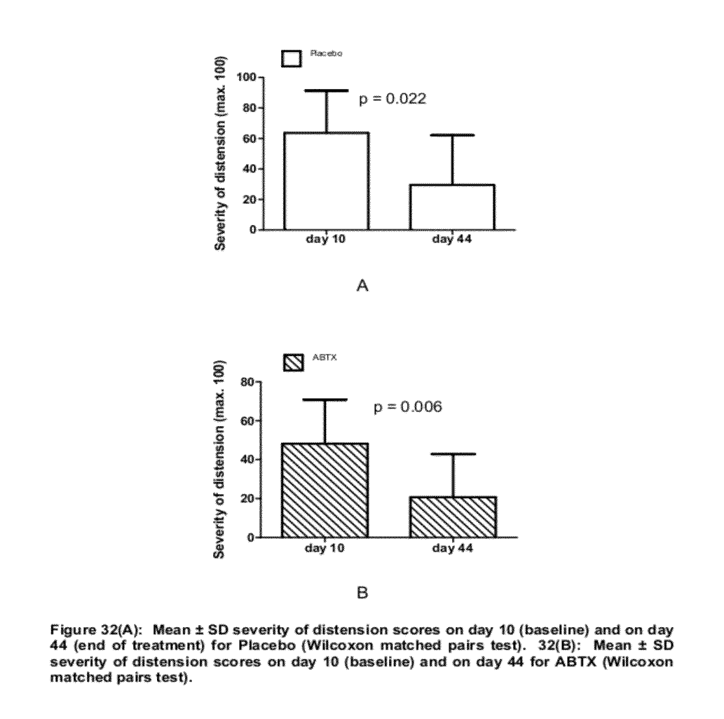

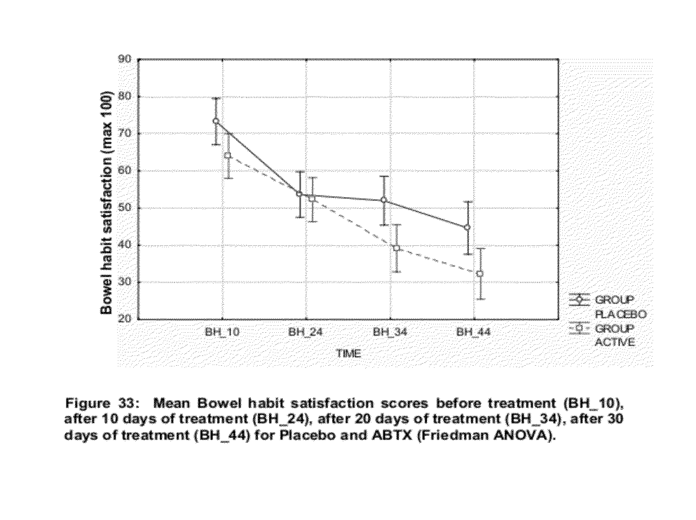

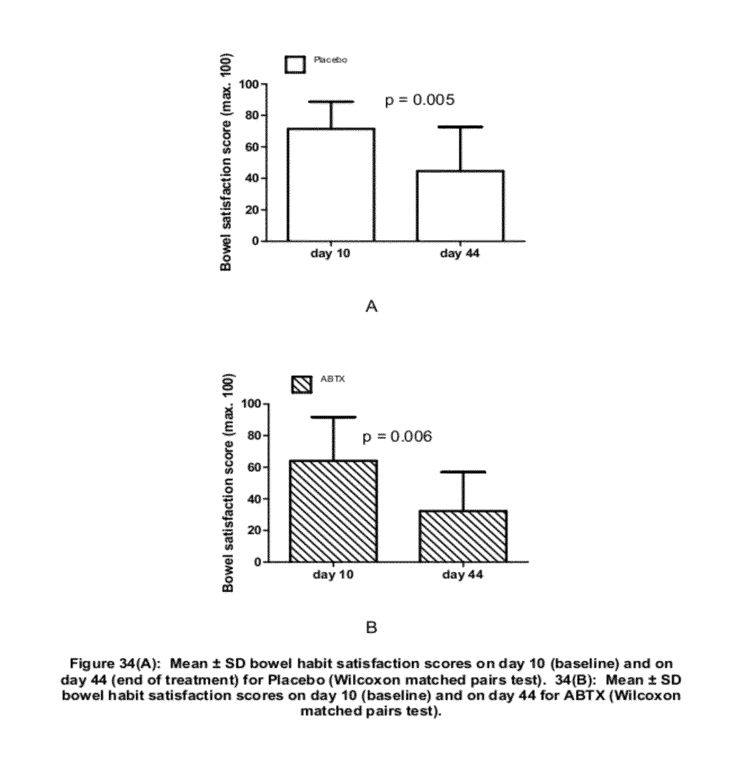

Although both groups have shown severity of distension reductions from baseline (FIG. 32), potentiated clinoptilolite “C35” had a significant less severity mean on day 34 compared to Placebo. Unfortunately, this effect was not sustained, as subsequent measurement (day 44, after 30 days of treatment) revealed no significance between Placebo and potentiated clinoptilolite “C35”. (Table 6.8).

The current trial showed that this IBS population were largely unsatisfied with their bowel habit prior to treatment (Table 6.9). The baseline scores dropped significantly across treatment weeks in the PP population. Furthermore, ITT analysis revealed significant differences between day 10 and day 44, in both the Placebo and potentiated clinoptilolite “C35” group. At the end of treatment patients from both groups were much more satisfied with their bowel habits. In addition, no significant difference was observed between Placebo and potentiated clinoptilolite “C35”.

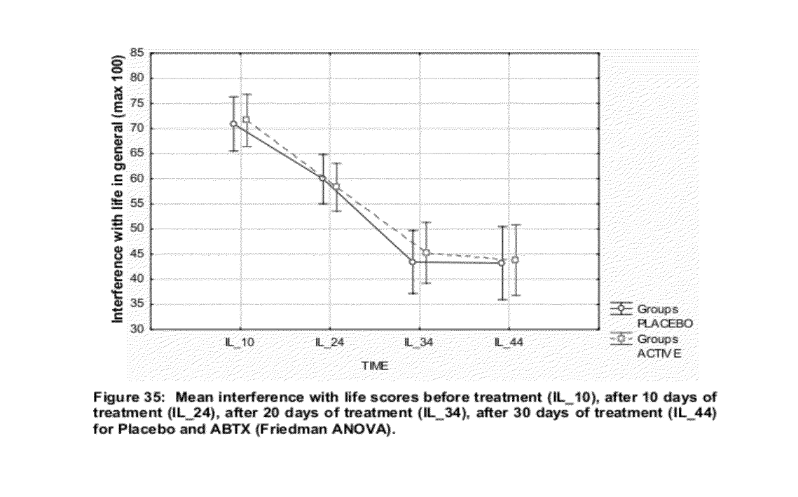

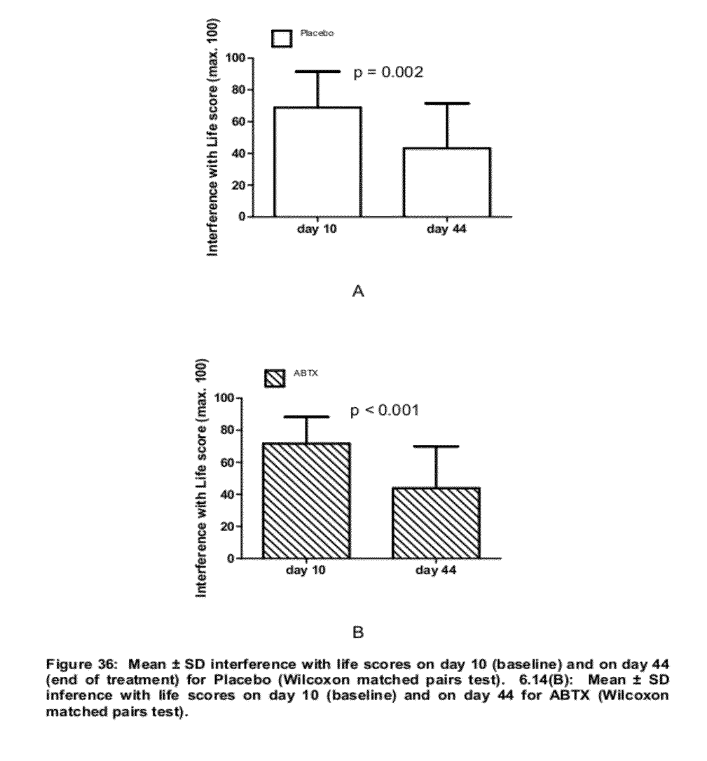

The “interference with life in general” scores were quite high in this IBS population. However, the impact of IBS on participants' lives gradually decreased across the 4 weeks of treatment, in both the Placebo and potentiated clinoptilolite “C35” group (FIG. 35, PP). Compared with baseline (day 10) the end of treatment “interference” was significantly lower in both treatment groups (FIG. 36, ITT). For this reason no significant differences were observed between treatment groups on any of the assessment days (Table 6.10).

It was found that the mean stool consistency was not a reliable parameter [scored on categorical scale from 0—very hard to 5—very loose, scoring 3—smooth (normal) stool, was regarded as the optimal score; the mean for each week was calculated] in the current trial, since Placebo and potentiated clinoptilolite “C35” had significant differences during week one (baseline) (Table 6.11). This may be attributed to the small sample size that led to an unequal distribution of bowel sub typing in each treatment group. The placebo group had more constipation predominant patients than the potentiated clinoptilolite “C35” group. In addition; the Placebo group had none diarrhoea predominant patients. It is therefore likely that the Placebo group could have had a lower mean stool consistency compared to potentiated clinoptilolite“C35” at week one (baseline). As the patients entered the treatment weeks, the differences subside as both groups mean stool consistency increased gradually. The outcome was that no differences were observed between treatment groups in any of the 4 treatment weeks.

The proportion smooth stool consistency was also evaluated (number of smooth stool cases over total bowel movement cases). During treatment both treatment groups had a greater proportion compared to baseline. However, potentiated clinoptilolite “C35” had a significantly greater proportion of smooth stool compared to Placebo during the treatment weeks.

In terms of urgency, similar findings during baseline were observed between treatment groups. No significant differences was observed between potentiated clinoptilolite “C35” and Placebo on any of the 4 weeks of treatment.

The mean number of bowel movements per day did not change from baseline in either of the treatment weeks. Moreover, there was no significant difference observed between the potentiated clinoptilolite “C35” and Placebo group during any of the treatment weeks.

The proportion of days with no bowel movement did not differ significantly between treatment groups on any of the treatment weeks (Table 6.14). In the same way, the proportion of days with more than 3 bowel movements did also not differ between the potentiated clinoptilolite “C35” and Placebo on any of the treatment weeks (Table 6.15).

Heartburn and other gut symptoms have been reported in 25% to 50% of IBS patients and it is further documented that IBS patients are more likely to suffer from GORD than healthy controls. Neutacid™ is a trademark that consists of the natural zeolite, clinoptilolite, and is registered in Cuba as an antacid. It was, therefore; propose that potentiated clinoptilolite “C35” (with similar properties as Neutacid™) may reduce the incidence of heartburn episodes.

The mean proportion of heartburn days during treatment decreased significantly compared to baseline, in the Placebo group (Table 6.16). To the contrary, different from what has initially been anticipated, no significant difference was observed from baseline to treatment, in the ABTX group (Table 6.17). Furthermore, no significant difference was seen between Placebo and potentiated clinoptilolite “C35”, during treatment. At baseline, the mean proportion of days with heartburn was relatively low in the potentiated clinoptilolite“C35” group. Although not significantly lower than the Placebo group, it might have happen that a floor effect have occurred in the active treatment group, in which mild symptoms leave little room for detecting an improvement.

Although it is unable to confirm, the question that was used to assess these symptoms [“Did you experience any episodes of heartburn or indigestion (dyspepsia) today”], may have been hard to interpret correctly, by the participant.

In addition, these findings must be viewed with caution, as heartburn and dyspepsia are not the same conditions, in fact a consensus states that if gastro-oesophageal reflux symptoms (e.g. heartburn and acid regurgitation) is present concomitantly with dyspepsia the differential diagnosis should rather favour the GORD. In contrast, there is evidence that symptoms (listed in parenthesis) of dyspepsia (early satiety, vomiting, nausea, pain, bloating), GORD (heartburn, bloating, regurgitation), chronic constipation (gas, bloating) and IBS (abdominal pain, bloating, constipation, diarrhoea) may all coincide with one another

The End-of-study marketing potential (EMP) questionnaire is a novel questionnaire that was used to address global impression of the treatment received, in which participants were asked whether they would recommend the treatment to other IBS sufferers and whether they will use the product in the near future. The questionnaire was administered at the end of treatment and relied solely on the participant's perception of treatment. As far as known, this type of questionnaire has never been utilised before in IBS pilot trials. Although not statistical significant, a greater proportion of potentiated clinoptilolite “C35” participants compared to Placebo reported that they would recommend the treatment to other IBS sufferers and would use the product in the near future.

Both treatment groups have used mebeverine and lactulose, however; no loperamide was used during trial treatment. No significant difference between potentiated clinoptilolite “C35” and

Placebo with regards to rescue medication consumption was observed. Therefore, potentiated clinoptilolite “C35” was not characterised with less rescue medication consumption. Although not statistically analysed, the potentiated clinoptilolite “C35” group were characterised with more lactulose consumption compared to Placebo. In addition, participants from the Placebo group consumed more mebeverine during the treatment weeks.

Although there were no statistical significant differences in the proportion of patients experiencing at least one AE, it must be noted that participants in the Placebo group were 10% more likely to experience an AE compared to potentiated clinoptilolite “C35”. Furthermore, the patient with the highest score for the maximum proportion of days with AE came from the Placebo group (section 6.10).

This study managed to keep the dropouts relatively low, i.e. 12.12% (4/33). This is in concordance with literature recommendations.

Participants were equal distributed, in terms of duration and severity of symptoms, between the two treatment groups. Participants from the potentiated clinoptilolite “C35” group reported better global response to treatment compared to Placebo (i.e. more overall responserds came from the potentiated clinoptilolite “C35” group), but did not reach statistical significance. During week 3 and 4 of treatment a statistical significant proportion of potentiated clinoptilolite “C35” participants reported having “adequate relief” compared to Placebo. With regards to separate symptom parameters (pain, distension, bowel habit, interference with life in general), both potentiated clinoptilolite “C35” and Placebo groups showed significant improvements compared with baseline observations. After 20 days of treatment potentiated clinoptilolite “C35” had less severity of distension compared to Placebo, however; subsequent assessment revealed that this effect was not sustained from day 34 (day 20 of treatment) and onwards. Stool parameter (consistency, urgency, frequency) analysis did not reveal any differences between potentiated clinoptilolite “C35” and Placebo, but the active group was associated with a greater proportion of smooth stool passage than the Placebo. In addition, heartburn were left untreated within the potentiated clinoptilolite “C35” group, while participants from Placebo had less heartburn episodes when compared with baseline. A greater proportion of patients from potentiated clinoptilolite “C35” group considered the use of allocated treatment in future (statistically not significant). Likewise, a greater proportion of patients from the potentiated clinoptilolite “C35” reported that they will recommend the treatment to other IBS sufferers (statistically not significant). potentiated clinoptilolite “C35” was not associated with less frequent rescue medication consumption as no differences were noted between the two groups. Drug compliance and adverse events were similar between the two groups.

The placebo effect was for the most part of the trial present. This phenomenon is widely discussed in subjective disorders, like depression and pain, but also in neurodegenerative disorders like Parkinson's disease. IBS is no exception as previous trials have reported high response to placebos in 40% to 70% of patients. The reasons for the placebo effect is rarely known, but higher cognitive functions such as the endogenous endorphins may be a role player in the evolution of this common phenomenon.

In the current trial, guidelines were followed to keep the placebo response as small as possible, however it was largely unavoidable as a crude form of treatment is given (e.g. counselling, reassurance and cognitive behavioural treatment) in each and every trial, regardless of the treatment that is received. In addition, if this kind of reassurance is not provided, it might eventually lead to larger drop-outs. According to research, many predictors of high placebo response exist in IBS trials. The type of diagnostic criteria has been shown to be a predictor of high placebo response; Manning criteria have shown larger placebo responses compared to Rome criteria. IBS candidates in this trial were diagnosed according to the Rome criteria and therefore this problem should have been successfully overstepped. A greater number of office visits were found to decrease the placebo response.

According to German researchers, women in IBS trials are more prone to placebo response than men, this may further explain the high placebo response seen in this particular study, since this trial have only successfully enrolled one male patient.

Participants in this trial were instructed to use potentiated clinoptilolite C35/Placebo, one capsule three times daily; this might have had a significant contribution to the placebo response, as the placebo response increases with frequency of intervention. Furthermore, an adequate run-in phase was used and laboratory testing was done before randomisation, all in concordance with guidelines to minimise the placebo response. Some authors' recommend that IBS trials should be designed over long periods (longer than 3 months) to allow the placebo response to recede; interestingly, the duration of treatment had no effect on the placebo response.

This clinical trial pilot study have shown that IBS patients receiving potentiated clinoptilolite “C35” have a greater tendency towards improvement in terms of global symptom endpoints, compared to the control. Although, the placebo effect have been largely constant throughout the trial and little (if any) significance were observed between two groups in terms of separate symptom ratings (pain, distension, bowel habit, interference), difference was; however, seen in the severity of distension after 20 days (Table 6.8) of treatment. Furthermore, the proportion of patients reporting adequate relief in the potentiated clinoptilolite “C35” group reached significance after 3 weeks (i.e. 21 days) of treatment.

### Chapter 6.1

TABLE 6.1
Summary of demographics and baseline
characteristics of study patients

| | Absorbatox ™ C35 | Placebo |
|---|---|---|
| | (n = 15) | (n = 16) |
| Age | 45.93 ± 11.68 | 32.63 ± 13.41 |
| Gender (male:female) | 1:15 | 0:16 |
| Race (n) | | |
| White | 15 | 16 |
| Coloured | 1 | 0 |
| Black | 0 | 0 |
| BMI (mean ± SD) | 28.69 ± 6.33 | 26.67 ± 7.38 |
| Family history of IBS (%) | 80 | 50 |
| ROME III bowel | | |
| classification (n) | | |
| IBS-C | 3 | 6 |
| IBS-D | 4 | 0 |
| IBS-M | 8 | 10 |
| Symptoms (mean ± SD) | | |
| Duration of symptoms (years) | 15.98 ± 11.47^{ns} | 14.06 ± 11.92 |
| IBS TOTAL Severity | 271.89 ± 70.73^{ns} | 298.00 ± 79.04 |
| score (max 500) | | |
| Pain/discomfort^{#} | 42.13 ± 22.92^{ns} | 43.43 ± 27.64 |
| Distension^{#} | 48.16 ± 22.64^{ns} | 63.63 ± 27.75 |
| Satisfaction with | 64.01 ± 27.66^{ns} | 71.47 ± 17.22 |
| bowel habit^{#$} | | |
| IBS interference | 71.59 ± 16.69^{ns} | 68.84 ± 22.73 |
| with life^{#}* | | |
| Mean number of stools per day | 1.43 ± 0.86^{ns} | 1.42 ± 0.99 |
| Medication usage (%) | | |
| Antacids/PPI | 20 | 18.75 |
| CAM users | 46.67 | 25 |
| Zelnorm ® (previously used) | 26.67 | 0 |
| Laxatives | 20 | 25 |
| Antidiarrhoeals | 6.67 | 0 |
| Antispasmodics | 66.67 | 50 |

^{#}maximum = 100, scored on visual analogue scales
^{$}0 = completely satisfied; 100 = completely unsatisfied
*0 = no interference at all; 100 = completely interfering
^{ns}no statistically significant differences were noted between treatment groups (p > 0.05).

TABLE 6.3
Mean total severity scores on day 10, 24, 34, 44 for Placebo
and Absorbatox ™ C35 (Mann-Whitney U test)

| | | Mean total | | |
|---|---|---|---|---|
| Assessment | | severity | | |
| Interval (day) | Group | scores | SD | p-value |
| 10 | Placebo | 298.00 | 79.04 | 0.247 |
| | ABTX | 271.9 | 70.73 | |
| 24 | Placebo | 205.3 | 115.7 | 0.953 |
| | ABTX | 201.7 | 98.74 | |
| 34 | Placebo | 179.6 | 106.3 | 0.371 |
| | ABTX | 140.2 | 82.08 | |
| 44 | Placebo | 155.6 | 131.20 | 0.777 |
| | ABTX | 128.6 | 93.87 | |

TABLE 6.4
Percentage of participants currently suffered from abdominal
pain on day 10, 24, 34, 44 [Fisher exact (one tailed) test].

| | | | | | p-value |
|---|---|---|---|---|---|
| | Absorbatox ™ C35 | | Placebo | | (Fisher-exact, one |
| Days | % (n) | n | % (n) | n | tailed) |
| 10 | 93.33 | 15 | 93.75 | 16 | 0.742 |
| 24 | 80.00 | 15 | 81.25 | 16 | 0.641 |
| 34 | 66.67 | 15 | 64.29 | 14 | 0.600 |
| 44 | 60.00 | 15 | 57.14 | 14 | 0.587 |

TABLE 6.5
Mean Severity of pain scores on day 10, 24, 34, 44 for Placebo
and Absorbatox ™ C35 (Mann-Whitney U test)

| | | Mean total | | |
|---|---|---|---|---|
| Assessment | | severity | | |
| Interval (day) | Group | scores | SD | p-value |
| 10 | Placebo | 43.43 | 27.64 | 0.890 |
| | ABTX | 42.13 | 22.92 | |
| 24 | Placebo | 18.30 | 32.20 | 0.126 |
| | ABTX | 25.17 | 28.72 | |
| 34 | Placebo | 20.24 | 28.98 | 0.809 |
| | ABTX | 18.55 | 21.45 | |
| 44 | Placebo | 16.09 | 28.99 | 0.244 |
| | ABTX | 18.45 | 21.42 | |

TABLE 6.6
Mean Number of days with pain (max. 10, rated as number
of days with pain in past 10 days) on day 10, 24, 34, 44
for Placebo and Absorbatox ™ C35 (Mann-Whitney U test)

| | | Mean total | | |
|---|---|---|---|---|
| Assessment | | severity | | |
| Interval (day) | Group | scores | SD | p-value |
| 10 | Placebo | 5.06 | 3.32 | 0.735 |
| | ABTX | 4.60 | 2.47 | |
| 24 | Placebo | 3.44 | 2.988 | 0.811 |
| | ABTX | 3.00 | 2.54 | |
| 34 | Placebo | 2.57 | 2.93 | 0.771 |
| | ABTX | 2.00 | 2.17 | |
| 44 | Placebo | 2.21 | 3.09 | 0.648 |
| | ABTX | 1.33 | 2.06 | |

TABLE 6.7
Percentage of participants currently suffered
from abdominal distension on day 10, 24, 34, 44

| | | | | | p-value |
|---|---|---|---|---|---|
| | Absorbatox ™ C35 | | Placebo | | (Fisher-exact, one |
| Days | % (n) | n | % | n | tailed) |
| 10 | 93.33 | 15 | 93.75 | 16 | 0.742 |
| 24 | 66.67 | 15 | 62.50 | 16 | 0.553 |
| 34 | 53.33 | 15 | 64.33 | 14 | 0.413 |
| 44 | 53.33 | 15 | 42.86 | 14 | 0.424 |

TABLE 6.8
Mean Severity of distension scores on day 10, 24, 34, 44 for Placebo
and Absorbatox ™ C35 (Mann-Whitney U test)

| | | Mean | | |
|---|---|---|---|---|
| | | interference | | |
| Assessment | | with life | | |
| Interval (day) | Group | scores | SD | p-value |
| 10 | Placebo | 63.63 | 27.75 | 0.082 |
| | ABTX | 48.16 | 22.64 | |
| 24 | Placebo | 40.85 | 33.44 | 0.692 |
| | ABTX | 36.04 | 25.17 | |
| 34 | Placebo | 38.25 | 29.66 | 0.024 |
| | ABTX | 17.20 | 18.71 | |
| 44 | Placebo | 29.55 | 32.55 | 0.553 |
| | ABTX | 20.72 | 22.17 | |

TABLE 6.9
Mean Bowel habit satisfaction scores on day 10, 24, 34, 44 for Placebo
and Absorbatox ™ C35 (Mann-Whitney U test).

| | | Mean Bowel | | |
|---|---|---|---|---|
| | | habit | | |
| Assessment | | satisfaction | | |
| Interval (day) | Group | scores | SD | p-value |
| 10 | Placebo | 71.47 | 17.22 | 0.540 |
| | ABTX | 64.01 | 27.66 | |
| 24 | Placebo | 52.31 | 21.46 | 0.812 |
| | ABTX | 52.22 | 23.20 | |
| 34 | Placebo | 51.97 | 20.46 | 0.176 |
| | ABTX | 39.17 | 27.91 | |
| 44 | Placebo | 44.62 | 28.13 | 0.176 |
| | ABTX | 32.27 | 24.70 | |

TABLE 6.10
Mean Interference with Life scores on day 10, 24, 34, 44 for Placebo
and Absorbatox ™ C35 (Mann-Whitney U test).

| | | Mean total | | |
|---|---|---|---|---|
| Assessment | | severity | | |
| Interval (day) | Group | scores | SD | p-value |
| 10 | Placebo | 68.84 | 22.73 | 0.857 |
| | ABTX | 71.59 | 16.69 | |
| 24 | Placebo | 59.51 | 18.70 | 0.857 |
| | ABTX | 58.31 | 18.55 | |
| 34 | Placebo | 43.43 | 24.38 | 0.896 |
| | ABTX | 45.27 | 22.61 | |
| 44 | Placebo | 43.22 | 28.33 | 0.983 |
| | ABTX | 43.82 | 26.15 | |

TABLE 6.11
Mean stool consistency scores for week 1-6 [scored on categorical scale:
0—very hard to 5—very loose (watery)] (Mann-Whitney test u test).

| | | Absorbatox ™ | | | | p-value |
|---|---|---|---|---|---|---|
| | | C35 | | Placebo | | Mann-Whitney |
| | Week | Mean | SD | Mean | SD | U test |
| Baseline | 1 | 2.75 | 0.89 | 1.96 | 0.76 | 0.011 |
| | 2 | 2.77 | 1.02 | 2.07 | 0.58 | 0.060 |
| Treatment | 3 | 2.63 | 0.75 | 2.22 | 0.61 | 0.127 |
| | 4 | 2.59 | 0.66 | 2.35 | 0.78 | 0.428 |
| | 5 | 2.68 | 0.54 | 2.38 | 0.88 | 0.176 |
| | 6 | 2.86 | 0.39 | 2.53 | 0.75 | 0.196 |

TABLE 6.12
Mean urgency scores between treatment groups for week 1-6 [rated
on categorical scale; 0—no urgency at all; 5—very severe urgency]
(Mann-Whitney U test).

| | | | | | | p-value |
|---|---|---|---|---|---|---|
| | | Absorbatox ™ | | | | Mann- |
| | | C35 | | Placebo | | Whitney U |
| | Week | Mean | SD | Mean | SD | test |
| Baseli | 1 | 2.10 | 0.70 | 1.87 | 0.68 | 0.513 |
| | 2 | 2.31 | 1.00 | 2.10 | 0.60 | 0.634 |
| Treatment | 3 | 1.99 | 0.72 | 1.98 | 0.47 | 0.827 |
| | 4 | 2.02 | 0.57 | 1.93 | 0.54 | 0.677 |
| | 5 | 2.05 | 0.61 | 2.19 | 0.76 | 0.467 |
| | 6 | 1.94 | 0.60 | 2.33 | 0.67 | 0.156 |

TABLE 6.13
Mean number of bowel movements per day between treatment
groups (Mann-Whitney U test).

| | | | | | | p-value |
|---|---|---|---|---|---|---|
| | | Absorbatox ™ | | | | Mann- |
| | | C35 | | Placebo | | Whitney U |
| | Week | Mean | SD | Mean | SD | test |
| Baseli | 1 | 1.46 | 0.78 | 1.44 | 1.10 | 0.6779 |
| | 2 | 1.40 | 0.94 | 1.40 | 0.87 | 0.858 |
| Treatment | 3 | 1.50 | 0.83 | 1.38 | 0.90 | 0.648 |
| | 4 | 1.36 | 0.74 | 1.34 | 0.66 | 0.937 |
| | 5 | 1.45 | 0.59 | 1.16 | 0.77 | 0.252 |
| | 6 | 1.52 | 0.59 | 1.23 | 0.60 | 0.228 |

TABLE 6.14
Mean proportion of days with no bowel movement (no stool; BM = 0) of
Placebo and Absorbatox ™ C35 (Mann-Whitney).

| | | | | | | p-value |
|---|---|---|---|---|---|---|
| | | Absorbatox ™ | | | | Mann- |
| | | C35 | | Placebo | | Whitney u |
| | Week | Mean | SD | Mean | SD | test |
| Baseli | 1 | 0.21 | 0.19 | 0.31 | 0.32 | 0.515 |
| | 2 | 0.22 | 0.58 | 0.27 | 0.27 | 0.584 |
| Treatment | 3 | 0.18 | 0.44 | 0.25 | 0.22 | 0.351 |
| | 4 | 0.22 | 0.94 | 0.22 | 0.22 | 0.984 |
| | 5 | 0.13 | 0.13 | 0.28 | 0.33 | 0.378 |
| | 6 | 0.09 | 0.24 | 0.17 | 0.22 | 0.502 |

TABLE 6.15
Mean proportion of days with more than 3 bowel movement (BM > 3)
of Placebo and Absorbatox ™ C35 (Mann-Whitney U test).

| | | | | | | p-value |
|---|---|---|---|---|---|---|
| | | Absorbatox ™ | | | | Mann- |
| | | C35 | | Placebo | | Whitney U |
| | Week | Mean | SD | Mean | SD | test |
| Baseli | 1 | 0.06 | 0.13 | 0.03 | 0.06 | 0.818 |
| | 2 | 0.06 | 0.15 | 0.04 | 0.08 | 0.931 |
| Treatment | 3 | 0.02 | 0.05 | 0.03 | 0.06 | 0.710 |
| | 4 | 0.03 | 0.06 | 0.01 | 0.04 | 0.309 |
| | 5 | 0.03 | 0.06 | 0.01 | 0.04 | 0.292 |
| | 6 | 0.01 | 0.04 | 0.01 | 0.03 | 0.620 |

TABLE 6.16
Mean proportion of days with heartburn/dyspepsia at baseline
and treatment phase for Placebo (paired t test)

| | Mean | SD | p-value |
|---|---|---|---|
| Baseline | 0.37 | 0.34 | 0.030 |
| Treatment | 0.21 | 0.22 | |

TABLE 6.17
Mean proportion of days with heartburn/dyspepsia
at baseline and treatment phase for Absorbatox ™ C35
(paired t test)

| | Mean | SD | p-value |
|---|---|---|---|
| Baseline | 0.19 | 0.29 | 0.393 |
| Treatment | 0.13 | 0.17 | |

TABLE 6.18
The distribution on number of times participants have used a particular
Rescue Medication in Placebo and Absorbatox ™ C35.

| | Placebo | Absorbatox ™ C35 |
|---|---|---|
| Loperamide 2 mg tablets | 0 | 0 |
| Lactulose Dry 20 g | 15 | 34 |
| Mebeverine 135 mg | 13 | 7 |

TABLE 6.19
Summary of adverse events.

| | Placebo | Absorbatox ™ C35 |
|---|---|---|
| | (n = 16) | (n = 15) |
| Mean proportion of days with adverse | 0.09 ± 0.15 | 0.02 ± 0.04 |
| event (Mean ± SD) | | |
| Maximum proportion of days with | 0.53 | 0.13 |
| adverse event reported | | |
| Adverse events that were reported | | |
| (at least once): | | |
| Gastrointestinal disorders (%): | | |
| Abdominal distension | 3 (18.75) | 0 |
| Abdominal discomfort | 1 (6.25) | 0 |
| Abdominal pain (spasm/cramps) | 3 (18.75) | 2 (12.25) |
| Burbing | 1 (6.25) | 0 |
| Constipation | 0 | 0 |
| Diarrhoea | 0 | 0 |
| Dry mouth | 0 | 1 (6.25) |
| Flatulence | 1 (6.25) | 1 (6.25) |
| Heartburn | 1 (6.25) | 0 |
| Loose stools | 0 | 2 |
| Nausea | 2 (12.25) | 2 (12.25) |
| Vomiting | 1 (6.25) | 0 |
| Urinary tract disorders: | | |
| Polyuria | 1 (6.25) | 0 |
| Nervous system disorders: | | |
| Dizziness | 0 | 1 (6.25) |
| Headache | 1 (6.25) | 1 (6.25) |
| Other: | | |
| Fever | 1 (6.25) | 0 |
| Flu-like symptoms | 0 | 2 (12.25) |

TABLE 6.20
Summary of primary outcome, secondary outcome and stool parameters
results in the ITT population (ns: not significant).

| | | | Method | | |
|---|---|---|---|---|---|
| | | p - values | of | | |
| Measurement | | (results) | analysis | Type of data | Comment |
| Primary Outcome: | | | | | |
| 1) | Overall responders | 0.085 | Fisher | ABTX vs. | ns |
| 2) | Weekly response to | | exact test | placebo | |
| | treatment: | | (one-tailed) | (unpaired) | |

| | | |
|---|---|---|
| Week one: | 0.578 | ns |
| Week two: | 0.422 | ns |
| Week three: | 0.02 | significant |
| Week four: | 0.016 | significant |

Secondary outcome:

| | |
|---|---|
| 1) | Total severity score (IBS-SSS) |

| | | | | |
|---|---|---|---|---|
| Placebo | 0.005 | Wilcoxon | Day 10 vs. day | significant |
| ABTX | <0.001 | signed rank | 44 (paired data) | significant |

| | |
|---|---|
| 50 points reduction in IBS- | According |
| SSS from day 10 to day 44? | to Francis |

| | | | |
|---|---|---|---|
| Placebo | ↓ 142.4 (298.0 − 155.6) | et al., | YES |
| ABTX | ↓ 143.3 (271.9 − 128.6) | 1997 | YES |

| Stool parameters: | | | |
|---|---|---|---|
| 1) | Stool consistency | Mann- | ABTX vs. |

| | | |
|---|---|---|
| Mean stool consistency | Whitney | Placebo |

| | | | | |
|---|---|---|---|---|
| Week one (baseline) | 0.011 | U test | (unpaired data) | Significant |
| Week two (baseline) | 0.060 | | | ns |
| Week three | 0.127 | | | ns |
| Week four | 0.428 | | | ns |
| Week five | 0.176 | | | ns |
| Week six | 0.196 | | | ns |

Proportion smooth stool

| | | | | |
|---|---|---|---|---|
| ABTX vs. Placebo | 0.049 | Mann- | ABTX vs. | significant |

| | | | |
|---|---|---|---|
| | | Whitney | Placebo |
| | | U test | (unpaired data) |
| 2) | Urgency | | |

| | | |
|---|---|---|
| Mean stool urgency | Mann- | ABTX vs. |

| | | | | |
|---|---|---|---|---|
| Week one (baseline) | 0.513 | Whitney | Placebo | ns |
| Week two (baseline) | 0.634 | U test | (unpaired data) | ns |
| Week three | 0.827 | | | ns |
| Week four | 0.677 | | | ns |
| Week five | 0.467 | | | ns |
| Week six | 0.156 | | | ns |

| | | | |
|---|---|---|---|
| 3) | Frequency | Mann- | Placebo vs. |

| | | |
|---|---|---|
| Mean number of BM/day | Whitney | ABTX |

| | | | | |
|---|---|---|---|---|
| Week one (baseline) | 0.678 | U test | (unpaired data) | ns |
| Week two (baseline) | 0.858 | | | ns |
| Week three | 0.648 | | | ns |
| Week four | 0.937 | | | ns |
| Week five | 0.252 | | | ns |
| Week six | 0.228 | | | ns |

| | | |
|---|---|---|
| Mean proportion of days | Mann- | Placebo vs. |
| with BM = 0 | Whitney | ABTX |

| | | | | |
|---|---|---|---|---|
| Week one (baseline) | 0.515 | U test | (unpaired data) | ns |
| Week two (baseline) | 0.584 | | | ns |
| Week three | 0.351 | | | ns |
| Week four | 0.984 | | | ns |
| Week five | 0.378 | | | ns |
| Week six | 0.502 | | | ns |

| | | |
|---|---|---|
| Mean proportion of days | Mann- | Placebo vs. |
| with BM > 3 | Whitney | ABTX |

| | | | | |
|---|---|---|---|---|
| Week one (baseline) | 0.818 | U test | (unpaired data) | ns |
| Week two (baseline) | 0931 | | | ns |
| Week three | 0.710 | | | ns |
| Week four | 0.309 | | | ns |
| Week five | 0.292 | | | ns |
| Week six | 0.620 | | | ns |

The research resulting in this invention has proven that clinopstilolite potentiated in the method covered by the invention has a “bio-mop” effect due to its ability to bind bioactive amines

1.-44. (canceled)

45. A method of treatment of symptoms associated with gastroesophageal reflux disease (GORD) comprising
orally administering to a patient in need thereof, a therapeutically effective amount of potentiated clinoptilolite.

46. The method of claim 45, wherein said potentiated clinoptilolite is administered to said patient at a dosage rate ranging from about 500 mg to 3000 mg in total per day.

47. The method of claim 46, further comprising administering said potentiated clinoptilolite to said patient one to three times over a single day, wherein the patient is a human or a non-human animal.

48. The method of claim 45, wherein said potentiated clinoptilolite is produced by a method comprising:
washing clinoptilolite in a saline solution having any one of a sodium content, a Potassium content, a Magnesium content a Calcium content, and combinations thereof, each ranging from about 0.1% to about 60% each, by means of repeated variable exposure to optimise displacement of naturally occurring elements in the said clinoptilolite, to provide a washed clinoptilolite fraction; and
drying the washed clinoptilolite fraction by air heated to a temperature ranging from about 40° C. to about 450° C.

49. A method of protecting a patient against non-steroidal anti-inflammatory drug induced ulcers comprising
orally administering to a patient in need thereof, a therapeutically effective amount of potentiated clinoptilolite.

50. The method of claim 49, wherein said potentiated clinoptilolite is administered to said patient at a dosage rate ranging from about 500 mg to 3000 g per dose.

51. The method of claim 50, further comprising administering said potentiated clinoptilolite to said patient one to three times over a single day, wherein the patient is a human or a non-human animal.

52. The method of claim 49, wherein said potentiated clinoptilolite is produced by a method comprising:
washing clinoptilolite in a saline solution having any one of a sodium content, a Potassium content, a Magnesium content, a Calcium content, and combinations thereof, each ranging from about 0.1% to about 60% each, by means of repeated variable exposure to optimise displacement of naturally occurring elements in the said clinoptilolite, to provide a washed clinoptilolite fraction; and drying the washed clinoptilolite fraction by air heated to a temperature ranging from about 40° C. to about 450° C.

53. A method of treatment of symptoms associated with irritable bowel syndrome comprising
orally administering to a patient in need thereof, a therapeutically effective amount of potentiated clinoptilolite.

54. The method of claim 53, wherein said potentiated clinoptilolite is administered to said patient at a dosage rate ranging from about 500 mg to 3000 g per dose.

55. The method of claim 54, further comprising administering said potentiated clinoptilolite to said patient one to three times over a single day, wherein the patient is a human or a non-human animal.

56. The method of claim 53, wherein said potentiated clinoptilolite is produced by a method comprising:
washing clinoptilolite in a saline solution having any one of a sodium content, a Potassium content, a Magnesium content, a Calcium content, and combinations thereof, each ranging from about 0.1% to about 60% each, by means of repeated variable exposure to optimise displacement of naturally occurring elements in the said clinoptilolite, to provide a washed clinoptilolite fraction; and drying the washed clinoptilolite fraction by air heated to a temperature ranging from about 40° C. to about 450° C.

57. A method of reducing levels of heavy metals in a patient comprising orally administering to a patient in need thereof, a therapeutically effective amount of potentiated clinoptilolite.

58. The method of claim 57, wherein said potentiated clinoptilolite is administered to said patient at a dosage rate ranging from about 500 mg to 3000 g per dose.

59. The method of claim 58, further comprising administering said potentiated clinoptilolite to said patient one to three times over a single day, wherein the patient is a human or a non-human animal.

60. The method of claim 57, wherein said potentiated clinoptilolite is produced by a method comprising:
washing clinoptilolite in a saline solution having any one of a sodium content, a Potassium content, a Magnesium content, a Calcium content, and combinations thereof, each ranging from about 0.1% to about 60% each, by means of repeated variable exposure to optimise displacement of naturally occurring elements in the said clinoptilolite, to provide a washed clinoptilolite fraction; and drying the washed clinoptilolite fraction by air heated to a temperature ranging from about 40° C. to about 450° C.

61. A method of treating veisalgia comprising
orally administering to a patient in need thereof, a therapeutically effective amount of potentiated clinoptilolite.

62. The method of claim 61, wherein said potentiated clinoptilolite is administered to said patient at a dosage rate ranging from about 500 mg to 3000 g per dose.

63. The method of claim 62, further comprising administering said potentiated clinoptilolite to said patient one to three times over a single day, wherein the patient is a human or a non-human animal.

64. The method of claim 61, wherein said potentiated clinoptilolite is produced by a method comprising:
washing clinoptilolite in a saline solution having any one of a sodium content, a Potassium content, a Magnesium content, a Calcium content, and combinations thereof, each ranging from about 0.1% to about 60% each, by means of repeated variable exposure to optimise displacement of naturally occurring elements in the said clinoptilolite, to provide a washed clinoptilolite fraction; and drying the washed clinoptilolite fraction by air heated to a temperature ranging from about 40° C. to about 450° C.
